(19) United States
(12) Patent Application Publication
(10) Pub. No.: US 2005/0261222 A1
(43) Pub. Date: Nov. 24, 2005

(54) Oligoribonucleotides for the treatment of irritative or inflammatory skin symptoms through RNA interference

(75) Inventors: Rainer Wolber, Hamburg (DE); Ludger Kolbe, Dohren (DE); Claudia Mundt, Bremen (DE); Stefan Gallinat, Wedel (DE); Ute Breitenbach, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/072,031

(22) Filed: Mar. 3, 2005

(30) Foreign Application Priority Data
Mar. 4, 2004 (DE) 10 2004 010 547.2

(51) Int. Cl. A61K 48/00; C07H 21/04

(52) U.S. Cl. 514/44; 536/23.1

(57) ABSTRACT

The invention is a double-stranded oligoribonucleotide or a physiologically compatible salt thereof, which is capable of inducing the degradation of mRNA of one of more structures involved in inflammation or irritation of the skin. The invention is also cosmetic or therapeutic compositions comprising one or more such double-stranded oligoribonucleotide and methods of treatment by use thereof.

### FIELD OF THE INVENTION

The invention concerns oligoribonucleotides which induce the degradation of mRNA in enzymes and structures involved in the inflammation process of the skin and which are particularly suitable for the prophylaxis and treatment of inflamed skin conditions and/or for skin protection with skin determined as sensitive. Furthermore, the invention concerns cosmetic or dermatological formulations, in particular cosmetic or dermatological formulations which provide specific care for the skin after sunbathing or shaving and which prevent afterreactions of the skin to the effects of UV radiation and/or irritation caused by shaving.

### BACKGROUND OF THE INVENTION

The skin, in particular the epidermis, is especially prone to external influences as a barrier organ of the human organism. According to current scientific understanding, the skin represents an immunological organ which, as an immunocompetent peripheral compartment, plays a unique role in inductive, effective and regulative immunoprocesses of the entire organism.

The epidermis is richly endowed with nerves and nerve endings such as Vater-Pacini lamellar corpuscles, Merkel cell-neurite complexes and free nerve endings for the sense of pain, cold, heat and itching.

For individuals with sensitive, tender or injured skin, a neuro-sensory phenomenon characterized by stinging can be observed. This “sensitive skin” differs from “dry skin” with thickened and hardened strata cornea.

Typical reactions of “stinging” with sensitive skin are reddening, tightening and burning of the skin as well as itching. “Stinging” phenomena can be regarded as disturbances to be treated cosmetically. However, more significant itching (in particular with significant skin itching occurring with atopic disorders as well as itching occurring with skin diseases) can also be designated as a serious dermatological disturbance or neuro-sensory phenomenon.

Typical disruptive neuro-sensory phenomena associated with the terms “stinging” or “sensitive skin” are skin reddening, tingling, prickling, tightness and burning of the skin as well as itchiness. They can be caused by stimulant environmental conditions—e.g. massage, the effect of (wash-active) surfactants, climatic influence such as sun, cold, dryness, but also heat, radiant heat and UV radiation, e.g. the sun.

In the “Journal of the Society of Cosmetic Chemists” 28, S.197-209 (May 1977), P. J. Frosch and A. M. Kligman describe a method for estimating the “stinging potential” of topically administered substances. Lactic acid and pyruvic acid are used as positive substances here for example. In measurements according to this method, however, amino acids, in particular glycine, were also determined as active in neuro-sensory terms (such substances are referred as to “stingers”).

According to previous findings, the presence of such a form of sensitivity to very definite substances varies from individual to individual. This means that a person who experiences “stinging effects” on contact with a substance, will very likely experience these again on every subsequent contact. Contact with other “stingers” can, however, occur perfectly normally without any reaction.

Many individuals who are more or less sensitive also have to endure erythematous skin symptoms when using certain deodorants or antiperspirants. Erythematous skin symptoms also occur as accompanying symptoms with certain skin diseases or irregularities. As an example, the typical skin rash in the symptoms of acne is regularly characterized by more or less significant reddening.

### SUMMARY OF THE INVENTION

It was therefore the task of the present invention to remedy the disadvantages of the prior art. In particular, active substances and preparations containing such active substances should be provided for cosmetic and dermatological treatment and/or prophylaxis of erythematous, inflamed, allergic or autoimmune reactive symptoms, in particular dermatoses, but also the symptom of “stinging”.

In addition to the positive effects of sunlight, such as the general feeling of well-being, the formation of vitamin D3 and acne treatment, there are also negative effects which are to be counteracted. If the skin is exposed to the sun or an artificial radiation source for too long, a significantly delimited reddening in contrast to the unexposed skin, erythema solare, develops after a latency period of 2 to 3 hours. In the sunburn which has arisen in this manner a distinction is drawn between:
1^{st} degree: Erythema (reddening, feeling of warmth)—Clears up after 2 to 3 days and disappears with increasing pigmentation at the same time;
2^{nd} degree: Blister formation—Blisters form on the skin accompanied by burning and itching, the epidermis necroses extensively; and
3^{rd} degree: Cell damage—Acute cell damage occurs, the body reacts with fever, the epidermis necroses extensively.
   The 2^{nd} and 3^{rd} degree are also referred to as dermatitis solare.

The formation of erythemas depends on the wavelength. The erythema range of UV-B lies between 280 nm and 320 nm.

Around 90% of the ultraviolet radiation reaching the earth consists of UV-A radiation with a wavelength between 320 nm and 400 nm. While UV-B radiation varies significantly depending on numerous factors (e.g. time of the year or day, degree of latitude), UV-A radiation remains relatively constant day in day out independent of the season, time of day or geographical factors. At the same time, the majority of UV-A radiation penetrates the living epidermis, while around 70% of the UV-B radiation is held back by the stratum corneum.

For a long time it was incorrectly assumed that long-wave UV-A radiation only exhibits a negligible biological effect and that UV-B radiation was correspondingly responsible for most of the damage caused to the human skin by sunlight. Meanwhile, it has been confirmed by numerous studies that UV-A radiation is far more dangerous than UV-B radiation in respect to triggering photodynamic, specifically phototoxic reactions and chronic changes to the skin. The harmful effect of UV-B radiation can also by intensified by UV-A radiation.

As the ways in which the various wavelength ranges of UV light regions contribute to skin changes caused by light have not been fully explained, it is nowadays increasingly assumed that preventative protection against both UV-A and UV-B radiation (for example through the application of light protection filter substances in the form of a cosmetic or dermatological formulation onto the skin) is of fundamental importance. Cosmetic or dermatological agents should, when applied in a thin layer onto the skin, protect the latter from the negative effects of sunlight.

A spell spent sunbathing is generally enjoyed by most people, while the detrimental factors are initially disregarded. Nevertheless, an awareness of the negative effects stemming from too intensive an exposure to sunlight has developed over the past few years, as a result of which a greater number of sun protection agents with greater protective effect are applied.

Sunburn or light erythema represent the acute symptoms of sunlight. In addition to the effects of UV-A radiation already described, the afterreaction of the skin also leads to reduced sebum production and a drying of the skin. Special active substances can therefore be used to sooth and care for the skin damaged by sunlight, such as fat replenishing and moisturizing agents, inflammation-relieving and cooling substances, locally anesthetizing substances and/or disinfectant substances, in order to prevent possible skin infections.

Inflammation-relieving or anti-inflammatory substances derived from plants such as azulene and bisabolol (chamomille), glycyrrhizic acid (liquorice root), hamamelin (hamamelis) or entire extracts, e.g. from aloe vera or chamomile, are used for example. These reveal a certain degree of success in mild cases and locally restricted erythema reactions. The same applies for creams with a high content of etheric oils or panthenol.

So-called after-sun preparations are intended to cool the skin after sunbathing and improve its moisturizing performance, whereby provision of the cooling effect plays a central role. This cooling effect is generally achieved through high amounts of ethanol, which spontaneously evaporates when applying the formulation to the skin. The drawback to these state-of-the-art formulations is nevertheless that a long-term regeneration of the skin cannot be achieved through mere cooling.

The task of the present invention was therefore to find cosmetic or dermatological preparations which do not manifest the disadvantages of the prior art and which, in particular, provide long-lasting skin care for skin damaged by ultraviolet radiation.

The growth of beard hair is triggered by increased formation of male hormones during puberty in growing men. Hormonal disturbances in women can also lead to form of beard growth which generally remains significantly behind that of male beard growth in its extent.

Shaving the face or other parts of the body covered with hair (such as the legs, armpits or pubic area) can be motivated by several constraints—e.g. of a religious or cultural nature; in the most straightforward case, hair growth is not desired by the person concerned for purely cosmetic reasons. Shaving is carried out either dry or wet. The development of new mechanical and electrical wet and dry shaving techniques nowadays enables a safe and thorough removal of the (beard) hair. With wet shaving, chemical aids—for example in the form of shaving gels, soaps or foams—are generally essential. These are required in order to soften the (beard) hair and hence minimize the effort required for cutting through—and consequently the unpleasant pulling on the hair shaft. Softening the (beard) hair is achieved through water absorption which is enabled by increasing the pH value of the hair. Wet shave agents therefore generally contain soap or fatty acid salts whose pH value lies in the range from 8-10. Products for wet shaving therefore produce a typical skin feeling which occurs after application. The skin feels dry and rough to the touch. This skin feeling is also referred to as a “squeaky-feeling” in the cosmetic industry and is extremely unpopular among consumers.

Cosmetic agents are also frequently recommended for dry shaving as well, so as to achieve as close a shave as possible, i.e. to cut the (beard) hair as closely to the skin surface as possible.

The skin parts affected by shaving can nevertheless not only be irritated by shaving aids, the mechanical irritation caused by shaving itself can also represent a stress to the skin which can lead to an unpleasant skin feeling (the so-called “shaving burn”).

A further task of the present invention was therefore to find cosmetic or dermatological preparations which are more effective in reducing the afterreactions of the skin to the (mechanical) irritation caused by shaving.

It was rather surprising, and herein lies the solution to these tasks, that the use of cosmetic or dermatological formulations with a content of interfering RNAs for the care of skin damaged by ultraviolet radiation and/or subjected to shaving stress as well as the alleviation of afterreactions of the skin to the effects of UV radiation and/or to the irritation of the skin caused by shaving, would remedy the disadvantages of the prior art.

The formulations for the purpose of the present invention are extremely satisfying preparations in every respect, which are distinguished by a long-lasting skin care effect. It was not to be predicted by the expert that the formulations used in accordance with the invention would provide better skin care for skin damaged by ultraviolet radiation or subjected to shaving stress, more effectively prevent the afterreactions of the skin to the effects of UV radiation and to the (mechanical) irritation caused by shaving, more effectively soothe skin irritated by sunbathing and shaving, cause mild sunburn and shaving burn to subside more quickly, more effectively promote skin smoothing, and that they are distinguished by an improved skin care effect and exhibit better sensory properties, such as application-friendliness to the skin or the absorption ability of the skin, than the preparations of the prior art.

It was also surprising that the preparations in the sense of the present invention also alleviate the afterreactions of the skin to the effects of UV radiation and to the (mechanical) irritation caused by shaving if they are used (i.e. applied to the skin) before or during a spell spent sunbathing and/or while shaving.

The invention is, of course, not restricted to preparations which are applied after the spell spent sunbathing and/or while shaving—by its nature it also extends to all cosmetic and dermatological applications in which a stress-alleviating effect is desired or could be advantageous.

The abovementioned positive effect of the formulations used in accordance with the invention equally applies to both skin damaged by UV radiation and skin subjected to shaving stress.

The skin irritations described above which are caused by ultraviolet light, shaving or a sensitive skin condition itself are brought about by so-called inflammation mediators. Two groups must be mentioned here above all: The arachidonic acid metabolites and proinflammatory cytokines. All these are designated as structures involved in the inflammation and/or irritation of the skin within the sense of this article. Arachidonic acid is a multiply unsaturated, long-chained fatty acid (20:4), which on irritation is released from membranes by the activation of a phospholipase A2. It then serves as a substrate for cyclooxygenases (COX) or lipoxygenases (LOX). The action of further activated enzymes finally causes the release of prostaglandins and leukotrienes (together referred to as eicosanoides), which activate the latter via specific receptors on the target cells. In the skin, both prostaglandins and leukotrienes trigger inflammatory reactions such as erythemas, edemas, migration of inflammatory cells and increased pain sensation. A wide range of medicines exists for inhibiting the release of prostaglandin, the best known being aspirin. However, all these attack at the same point, the cyclooxygenases. As the COX-1 also has important regulatory functions in the stomach and kidneys, it is important to preferentially inhibit the COX-2, an enzyme which is induced through inflammatory stimuli. Proinflammatory leukotrienes are formed by 5-lipoxygenase, an enzyme which can be activated through external stimuli via the protein FLAP (Five Lipoxygenase Activating Protein). Lipoxygenase inhibitors are still at an experimental stage. As there are a series of additional lipoxygenases which also have an anti-inflammatory effect in part, it is very important to develop inhibitors which have a high selectivity for the LOX-5. The second large group of proinflammatory proteins are the cytokines, proteins which activate autocrine or paracrine cells and hence trigger defense or protective functions or simply act chemotactically. Particularly important for the skin in this context are interleukin-1 alpha and beta (IL-1a, IL-1β) which is in part formed constitutively in the skin cells (IL-1a) or formed on a stimulus (IL-1β) or which is more intensely expressed (IL-1a), interleukin-6 (IL-6) which is expressed as a result of irritative stimuli, interleukin-8 (IL-8) which is released on skin irritations and then attracts inflammatory cells (and is therefore actually a chemokine) as well as tumor necrosis factor alpha (TNF-a), which can have more intense but also proapoptotic effects during inflammations. When treating inflammatory diseases, corticosteroids are primarily used for suppressing proinflammatory cytokines, with well-known side effects.

The inhibitors of prostaglandin production previously used in therapy are all inhibitors of enzyme activity. Active agents which inhibit the expression of enzymes are still in experimental trials. This route and the induction of natural antagonists (e.g. interleukin-1 receptor antagonist, IL-1RA) is being intensively researched for the reduction of proinflammatory cytokines. For the most part, the active agents examined involve substances which interfere with the signal transduction chain and hence suppress the protein expression.

Depending on where the signal transduction is interfered with and how specifically it succeeds, a series of more or less considerable drawbacks must be endured, as other routes of the signal cascade are also blocked unintentionally. These effects are at least responsible for part of the significant side effects of conventional therapy with corticosteroids.

### DETAILED DESCRIPTION OF THE INVENTION

A therapy using RNA interference, in which only the target molecule would be specifically inhibited in its expression, would therefore have considerable advantages.

Fire et al., Trends Genet. 15 (1999) 358-363 have revealed that the gene expression can be inhibited post-transcriptionally through the presence of double-stranded RNA fragments (dsRNA), which is homologous to the sequence of the mRNA of the gene examined, and designate this process as RNA interference (RNAi). The dsRNA affects the specific degradation of homologous mRNA in the cell in an as yet unexplained manner and thus inhibits the protein production.

WO 01/29058 discloses the identification of genes which are involved in RNAi, as well as their use for modulation of the RNAi activity. Elbashir et al., Nature 411 (2001) 494498, describe the specific expression inhibiting of endogenous and heterologous genes in various mammalian cells through short, interfering RNAs (short interfering RNAs, siRNAs). Double-stranded RNA fragments with a length of 21 nucleotides were used.

WO 01/68836 reveals the reduction of gene expression in cells by dsRNA. The dsRNA contains a nucleotide sequence which hybridizes at least a part of the gene to be inhibited under the physiological conditions of the cell with the nucleotide sequence. The dsRNA exhibits a length of 400 to 800 nucleotides.

WO 01/75164 discloses the use of dsRNA with a length of 21 to 23 nucleotides for the specific inactivation of gene functions in mammalian cells by RNAi.

Brummelkamp et al., Science 296 (2002) 550-553, describe a vector system which is to trigger the synthesis of siRNAs in mammalian cells and hence inhibit the gene expression of a target gene.

EP 1 214 945 A2 discloses the application of dsRNA with a length of 15 to 49 base pairs for inhibiting the expression of a specified target gene in mammalian cells. The dsRNA can be modified to increase their stability and are intended to enable the treatment of cancer, viral diseases and Morbus Alzheimer.

WO 02/053773 concerns an in vitro process for determining skin stress and skin aging in humans and animals, test kits and biochips suitable for realizing the process as well as a test procedure for demonstrating the effectiveness of cosmetic or pharmaceutical active substances against skin stress and skin aging.

Oligoribonucleotides which are suitable for the prophylaxis and treatment of undesired inflammations/irritations of the skin have so far not been described. A task of the present invention is the preparation of compositions which enable an effective treatment of and prophylaxis against irritations, inflammations and sensitive skin conditions, in particular against sunburn and shaving burn, without exhibiting the disadvantages of the prior art.

Hereinafter, the enzymes and proteins involved in inflammation of the skin will be referred to collectively as “proinflammatory proteins”.

This task is solved through oligoribonucleotides which are capable of inhibiting the gene expression of those enzymes and proteins which are involved in the inflammation process of the skin, in particular through double-stranded oligoribonucleotides or physiologically compatible salts thereof, which are capable of inducing the degradation of mRNA of one or more structures involved in the inflammation and/or irritation of the skin, in particular structures involved in the production of proinflammatory eicosanoids or cytokines. The terms eicosanoid-producing enzymes primarily refers to the enzymes (COX-2, LOX-5) involved in eicosanoid synthesis.

In addition to the oligoribonucleotides, physiologically compatible salts of such oligoribonucleotides are also suitable in accordance with the invention. For the sake of simplicity, the term oligoribonucleotide will be used in the following for both the oligoribonucleotides themselves as well as their salts, unless otherwise stated. The term oligoribonucleotide also includes modified oligoribonucleotides.

The enzymes preferably involved in the eicosanoid synthesis are the following oxygenases:
Prostaglandin G/H synthase-2—PGH2 Human (P35354) Prostaglandin G/H synthase 2 precursor (EC 1.14.99.1) (Cyclooxygenase-2) (COX-2) (Prostaglandin-endoperoxide synthase 2) (Prostaglandin H2 synthase 2) (PGH synthase 2) (PGHS-2) (PHS II). {GENE: PTGS2 OR COX2};
5-Lipoxygenase—LOX5 HUMAN (P09917) Arachidonate 5-lipoxygenase (EC 1.13.11.34) (5-lipoxygenase) (5-LO). {GENE: ALOX5 OR LOG5}—Homo sapiens (Human); and
5-Lipoxygenase activating Protein—FLAP HUMAN (P20292) 5-lipoxygenase activating protein (FLAP) (MK-886-binding protein). {GENE: ALOX5AP OR FLAP}.

Prostaglandin G/H synthase (PGHS-2) represents the step-controlling enzyme for prostaglandin synthesis. The enzyme 5-lipoxygenase is the step-controlling enzyme for leukotrien synthesis; FLAP activates the lipoxygenase and is therefore important for leukotrien formation. The numbers given represent accession numbers of the Swiss-PROT Database of the EMBL-EBI (European Bioinformatics Institute Heidelberg).

The other preferred structures which influence the irritation and inflammation of the skin include proinflammatory cytokines, in particular the following:
Interleukin-1a—1A HUMAN (P01583) Interleukin-1 alpha precursor (IL-1 alpha) (Hematopoietin-1). {GENE: IL1A};
Interleukin-1β—1L1B HUMAN (P01584);
Interleukin-1 beta precursor (IL-1 beta) (Catabolin) {GENE: 1L1B};
Interleukin-6—1L6 HUMAN (P05231);
Interleukin-6 precursor (IL-6) (B-cell stimulatory factor 2) (BSF-2) (Interferon beta-2) (Hybridoma growth factor) {GENE: 1L6 OR IFNB2};
Interleukin-8—IL8 HUMAN (P10145) Interleukin-8 precursor (IL-8) (CXCL8) (Monocyte-derived neutrophil chemotactic factor) (MDNCF) (T-cell chemotactic factor) (Neutrophil-activating protein 1) (NAP-1) (Lymphocyte-derived neutrophil-activating factor) (LYNAP) (Protein 3-10C) (Neutro-phil-activating factor) (NAF) (Granulocyte chemotactic protein 1) (GCP-1) (Emoctakin) {GENE: IL8}; and
T Tumor necrosis factor α—TNFA HUMAN (P01375) Tumor necrosis factor precursor (TNF-alpha) (Tumor necrosis factor ligand super-family member 2) (TNF-a) (Cachectin) {GENE: TNF OR TNFSF2 OR TNFA}.

The accession numbers of the Swiss-PROT Database of the EMBL-EBI (European Bioinformatics Institute Heidelberg) are given here.

Especially preferred are oligoribonucleotides which can inhibit the expression of cyclooxygenase, COX-2. Equally preferred are oligoribonucleotides which prevent the expression of interleukin 1a and β. Also preferred are oligoribonucleotides which can inhibit the mRNA expression of interleukins 6 and 8.

The oligoribonucleotides according to the invention represent RNA molecules (RNAs) which entirely or partially suppress the expression of these enzymes (gene silencing), which is presumably the result of degradation of the mRNA of one or more of the enzymes. This process is referred to as RNA interference (RNAi). The invention therefore involves oligoribonucleotides which can inhibit the mRNA degradation of structures involved in the irritation/inflammation of the skin. The mRNA whose degradation is to be effected is also referred to in the following as the target mRNA or target sequence. Correspondingly, the term target gene is used to refer to the gene and in particular the coding area of the gene whose expression is entirely or partially suppressed. If not otherwise indicated, the term target sequence refers to both the target gene and the target mRNA. The mRNA degradation of structures involved in the irritation/inflammation of the skin through RNAi proceeds sequence-specifically, i.e. an oligoribonucleotide generally only inhibits the expression of the corresponding target gene.

It is preferable if the oligoribonucleotide inhibits the expression of the gene of the structure involved in the inflammation and/or irritation of the skin by at least 30%, especially preferable by 50%.

The coding areas (cDNA) of the respective genes are preferred as the target sequence for the oligoribonucleotides according to the invention, including the 5′ and 3′ UTR areas. Especially preferred are the regions of the coding area which lie 50 to 100 nucleotides downstream of the start codon.

The oligoribonucleotides according to the invention preferably represent double-stranded RNA molecules (dsRNAs) which are homologous to the sequence of the target gene or a section thereof, i.e. correspond to the target gene in respect to the sense and anti-sense strand.

Homology is also indicated in accordance with the invention if the dsRNA is not fully identical to the target sequence. The oligoribonucleotides according to the invention exhibit, in relation to a length of 20 base pairs, preferably a maximum of 0 to 2, particularly preferred 0 to 1 and most preferably no variations from the target sequence deviations, i.e. a maximum of 0 to 2 and, in particular, a maximum of 0 to 1 base pairs are replaced by other base pairs.

The oligoribonucleotides according to the invention preferably have a length of 15 to 49 nucleotides, more preferable 17 to 30, particularly preferred 19 to 25 and most preferred a length of 20 to 23 nucleotides.

The object of the invention, however, is longer nucleotide fragments, such as dsRNAs which correspond to the respective target mRNAs or cDNAs in their length. These can typically be converted to fragments with a length of 21 to 23 nucleotides through soluble drosophila embryo extract (cf. WO 01/75164). Long-chained dsRNA also undergoes intracellular degradation to form short sections. Nevertheless, the direct use of long-chained dsRNA is generally not preferred, as this can cause an unspecific inhibition of the translation in mammalian cells.

The RNA duplexes according to the invention can exhibit smooth (blunt) or protruding (sticky) ends. Double-stranded oligoribonucleotides which exhibit an overhang of 1 to 6 at the 3′ end of each strand, preferably 1 or 2 nucleotides, have proven to be particularly effective.

The protruding nucleotides are preferably 2′ desoxynucleotides, particularly preferable 2′ desoxythymidine residues. Use of 2′ desoxynucleotides reduces the costs of RNA synthesis and increases the resistance of RNA to nuclease degradation. It is not essential for the protruding nucleotides to be the nucleotides homologous to the target sequence. They are not therefore taken into consideration in the deviations from the target sequence defined above. Oligoribonucleotides with short protrusions are especially preferred, in particular of 2 nucleotides, in which the protruding nucleotides of the anti-sense strand of the dsRNA are complementary to the target sequence.

Oligoribonucleotides which are homologous to such a section of the target gene and in particular to the corresponding double-stranded cDNA, whose sense strand is limited on the 5′ side by two adenosine residues (A) and on the 3′ side by two thymidine residues (T) or one thymidine and one cytidine residue (C), have proven to be particularly effective. The section limited by AA and TT or AA and TC preferably exhibits a length of 19 to 21, in particular 19 nucleotides and therefore has the general form AA(N₁₉₋₂₁)TT or AA(N₁₉₋₂₁)TC, whereby N represents a nucleotide. Further preferred are oligoribonucleotides which are complementary to a section of the target gene or to the corresponding double-stranded cDNA, which has the the general form AA(N₁₉) to AA(N₂₁). Particularly preferred for this are oligoribonucleotides which are homologous to the N₁₉₋₂₁ fragment of the areas cited. The especially preferred oligoribonucleotides thus exhibit a length of 19 to 21 base pairs, whereby the single-strands forming these oligoribonucleotides preferably exhibit on the 3′ side two additional 2′ desoxynucleotides each, in particular two 2′ desoxythymidine residues, so that the dsRNA comprises 19 to 21 base pairs and two protruding 2′ desoxynucleotides per string.

Should the target gene not contain any region of the form AA_{(N19-21)}, an attempt is made to find regions of the form NA_{(N1s-21)}, or any fragment of the form N₁₉₋₂₁. Although N₁₉₋₂₁ fragments which are limited by AA and TT, for example, are preferable, all dsRNA fragments which are homologous to the target sequence are suitable in accordance with the invention.

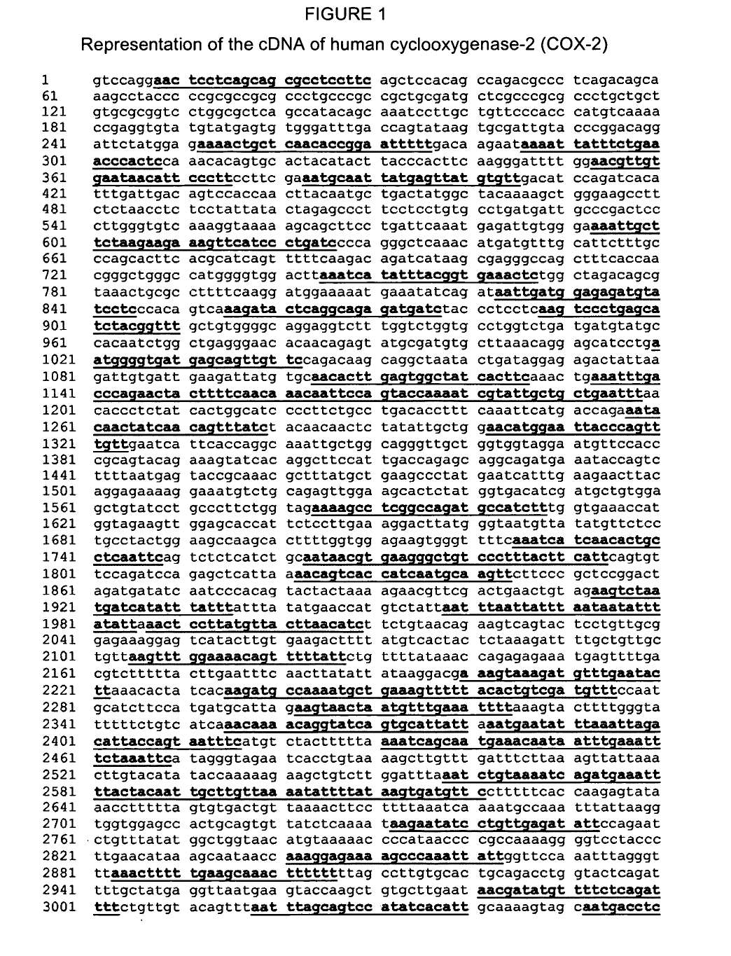

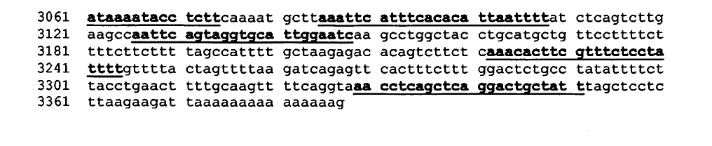

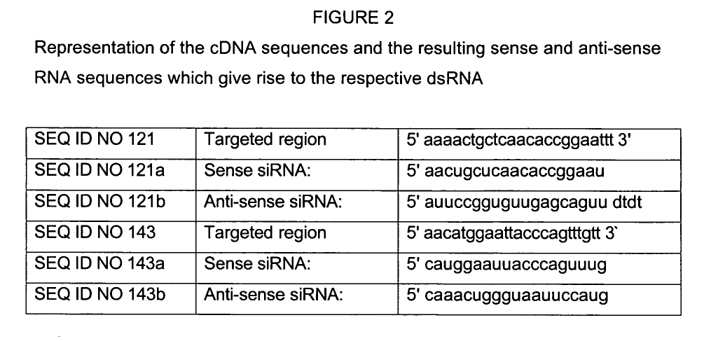

FIG. 1 shows the single-stranded cDNA of the cydoxigenase-2 in which all fragments of the form AA-N₁₉-TT and AA-N₁₉-TC are optically emphasized. As many of these sequences are overlaps, the emphasized areas are in part considerably longer than the individual fragments. FIG. 2 shows these fragments (targeted region) together with the corresponding homologous (sense RNA) and complementary (anti-sense RNA) RNA single-strands. Single-stranded RNAs which are modified on the 3′ side by two desoxythymidine residues (dt) are shown. The hybridization of two complementary single-stranded RNAs yields dsRNA with protruding 3′ ends which are formed through two 2′ desoxythymidine residues each.

The cycloxigenase-2 gene belongs to the preferred target genes for the oligoribonucleotides in accordance with the invention. Oligoribonucleotides which are homologous to the double-stranded sequence derived from the sequence shown in FIG. 1, sections thereof and, in particular, to the double-stranded sequences which are derived from the sections emphasized in FIG. 1, are correspondingly especially preferred in accordance with the invention. The double-stranded sequence derived from the sequence shown in FIG. 1 refers to the sequence which is formed from the sequence shown in FIG. 1 and the strand complementary to it. The other information is to be correspondingly understood.

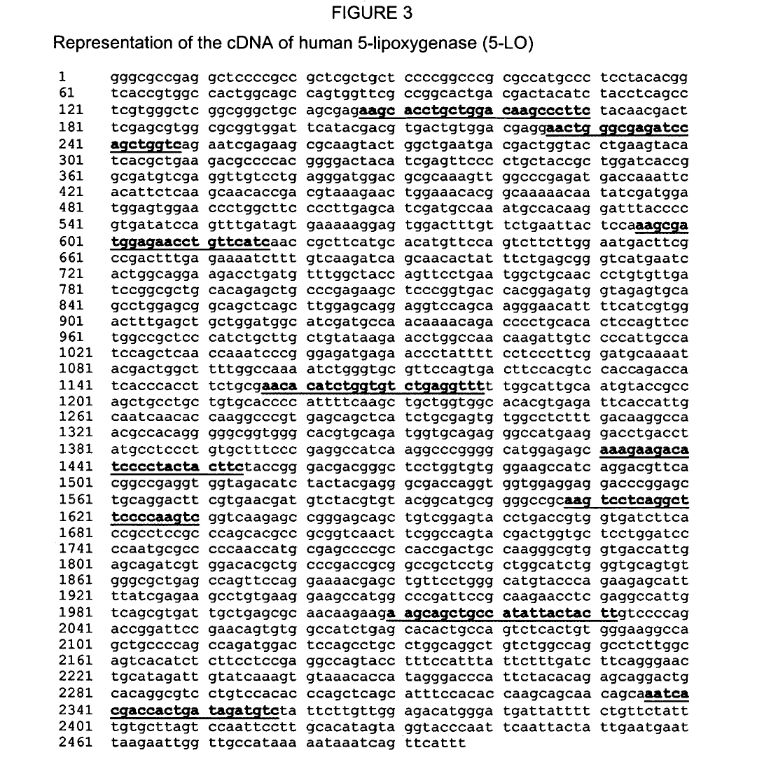

FIG. 3 shows the single-stranded cDNA of the 5-lipoxygenase (5-LOX). The preferred sequence regions, i.e. sequence regions with a length of 19 nucleotides which are flanked by AA and TT or TC, are also emphasized here. Oligoribonucleotides which are homologous to the double-stranded sequence derived from the sequence shown in FIG. 3, sections thereof and, in particular, to the double-stranded sequence which is derived from the region emphasized in FIG. 3, are preferred in accordance with the invention.

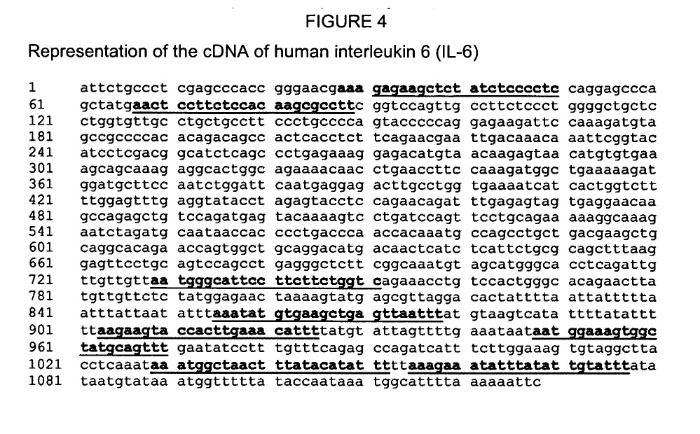

FIG. 4 shows the single-stranded cDNA of the interleukin-6 (IL-6), whereby preferred sequence regions are marked in turn. Oligoribonucleotides which are homologous to the double-stranded derived sequence derived from the sequence emphasized in FIG. 4, sections thereof and in particular to the double-stranded sequence which is derived from the regions emphasized in FIG. 4, are also preferred in accordance with the invention.

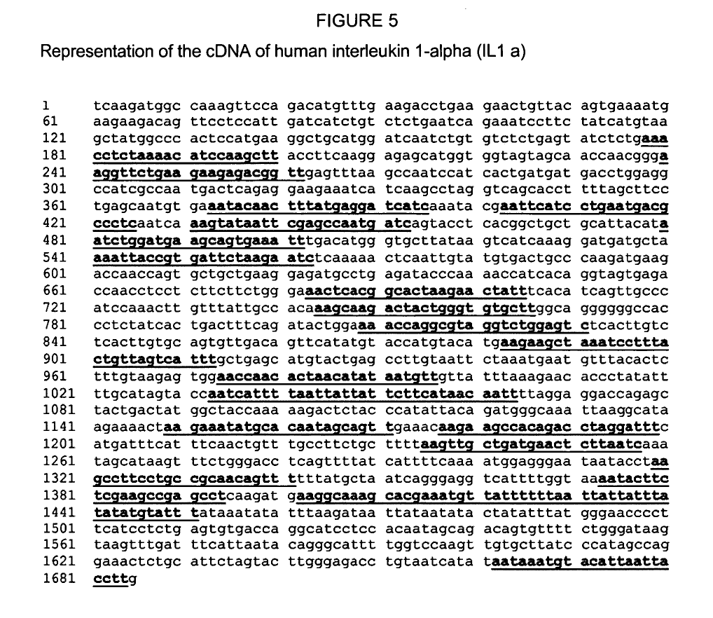

FIG. 5 shows the single-stranded cDNA of the interleukin-1 alpha (IL-1a), whereby preferred sequence regions are also marked. Oligoribonucleotides which are homologous to the double-stranded sequence derived from the sequence shown in FIG. 5, sections thereof and, in particular, to the double-stranded sequence which is derived from the regions emphasized in FIG. 5, are also preferred in accordance with the invention.

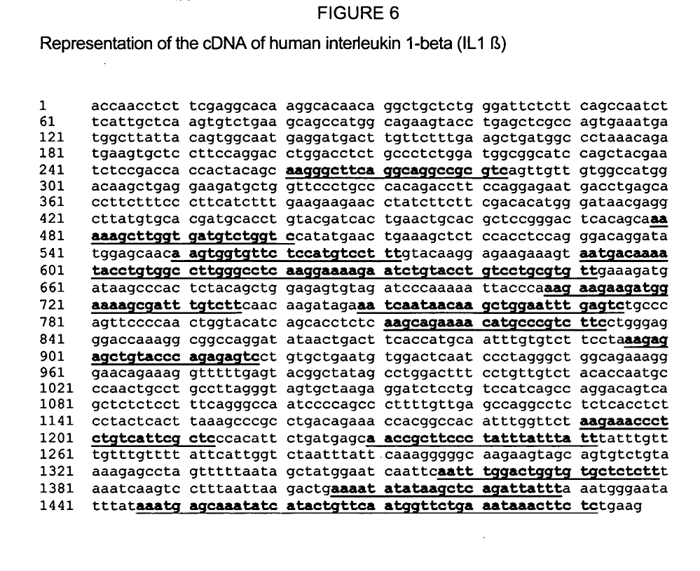

FIG. 6 shows the single-stranded cDNA of the interleukin-1 beta (IL-1β), whereby preferred sequence regions are also marked. Oligoribonucleotides which are homologous to the double-stranded sequence derived form the sequence shown in FIG. 6, sections thereof and, in particular, to the double stranded sequence which is derived from the regions emphasized in FIG. 6, are also preferred in accordance with the invention.

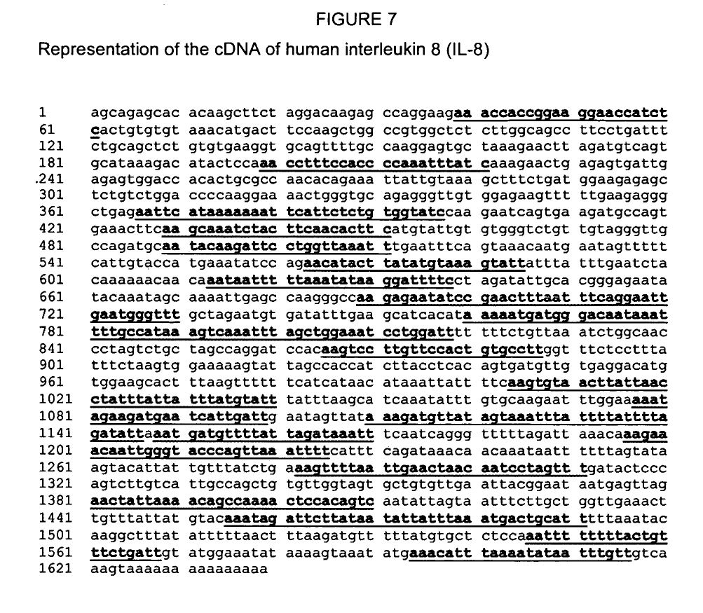

FIG. 7 shows the single-stranded cDNA of the interleukin-8 (IL-8), whereby the preferred sequence regions are also marked. Oligoribonucleotides which are homologous to the doubled-stranded sequence derived from the sequence shown in FIG. 7, sections thereof and, in particular, to the double-stranded sequence which is derived from the regions emphasized in FIG. 7, are also preferred in accordance with the invention.

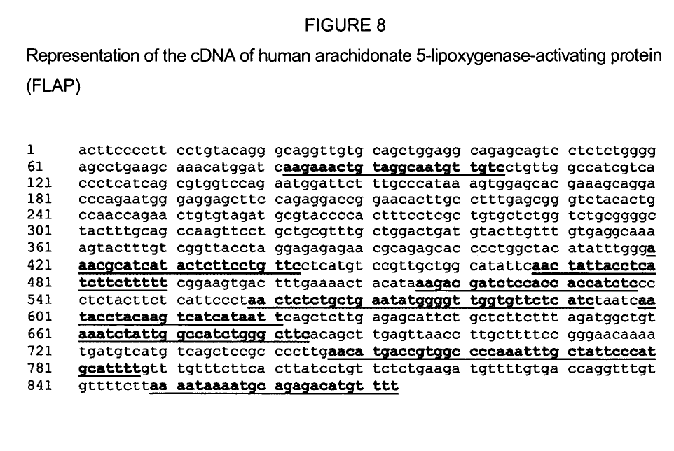

FIG. 8 shows the single-stranded cDNA of the 5-lipoxygenase-activating protein (FLAP), whereby preferred sequence regions are also marked. Oligoribonucleotides which are homologous to the double-stranded sequence derived from the sequence shown in FIG. 8, sections thereof and in particular, to the double-stranded sequence which is derived from the regions emphasized in FIG. 8, are also preferred in accordance with the invention.

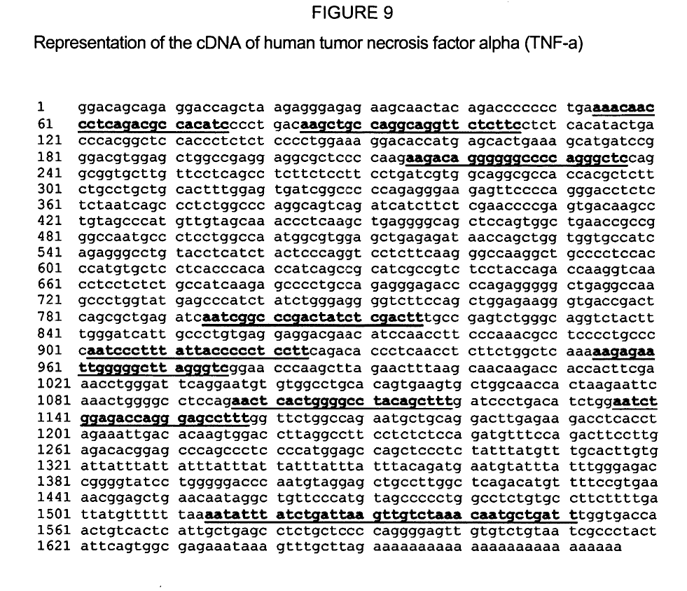

FIG. 9 shows the single-stranded cDNA of the tumor necrosis factor alpha (TNF-a), whereby preferred sequence regions are also marked. Oligoribonucleotides which are homologous to the double-stranded sequence derived from the sequence shown in FIG. 9, sections thereof and, in particular, to the double-stranded sequence which is derived from the regions emphasized in FIG. 9, are also preferred in accordance with the invention.

The oligoribonucleotides in accordance with the invention can also advantageously be integrated in the expression vectors, in particular those such which effect an expression of the oligoribonucleotides in mammalian cells. In this way, a stable inhibition of the expression of the target gene can be achieved even in the event of an intracellular degradation of the oligoribonucleotides, as oligoribonucleotides are continuously supplied subsequently through the vector-aided synthesis. One or more copies of a dsRNA can be integrated in a vector, or alternatively one or more copies each of two or more different dsRNAs. Suitable vector systems are described by Brummelkamp et al., op. cit. Preferred are mammalian expression vectors, in particular those such which contain a polymerase III H1-RNA promoter and 5 to 9 so-called loops which are formed from a dsRNA according to the invention and a sequence of the same length, which is reverse complementary to the dsRNA according to the invention and which serves as a spacer, as well as a termination signal of 5 consecutive thymidine residues. The vectors thus contain 5 to 9 copies of the respective dsRNA molecule. On this occasion, it can involve dsRNAs which are specific to 1 target gene, or alternatively dsRNAs which are specific to several different target genes.

The oligoribonucleotides according to the invention can be present in the form of unmodified oligoribonucleotides. However, this preferably involves oligoribonucleotides which can be chemically modified on the level of the sugar residues, the nucleobases, the phosphate groups and/or the skeleton located between these, for example in order to increase the stability of the oligoribonucleotides in cosmetic or dermatological preparations and/or in the skin, e.g. against a nucleolytic degradation, in order to improve the penetration of the oligoribonucleotides into the skin and the cells, in order to favorably influence the effectiveness of the oligoribonucleotides and/or to improve the affinity of the sequence sections to be hybridized.

Preferred are oligoribonucleotides in which one or more phosphate groups are replaced by phosphothioate, methylphosphonate and/or phosphoramidate groups, such as N3′-*P5′ phosphoramidate groups. Particularly preferred are oligoribonucleotides in which phosphate groups are replaced by phosphothioate groups. One or more of the phosphate groups of the oligoribonucleotide can be modified.

With a partial modification, terminal groups are preferably modified, although oligoribonucleotides in which all phosphate groups are modified are especially preferred, however. This also applies analogously for the modifications described in the following.

Preferred sugar modifications comprise the replacement of one or more ribose residues of the oligoribonucleotide by morpholine rings (morpholine oligoribonucleotides) or by amino acids (peptide oligoribonucleotides). Preferably all the ribose residues of the oligoribonucleotide are replaced by amino acid residues and, in particular, morpholine residues. 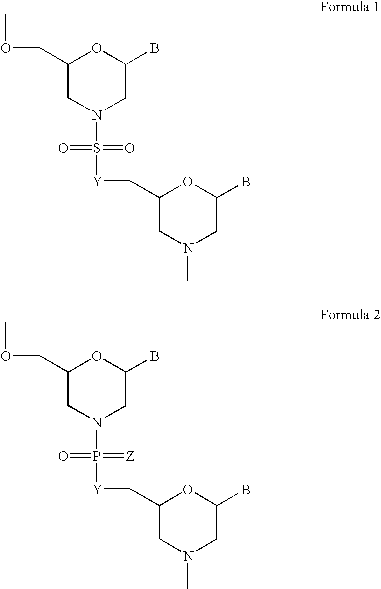
Especially preferred are morpholine oligoribonucleotides in which the morpholine residues are linked to each other via sulfonyl or preferably phosphoryl groups, as shown in Formula 1 or 2, wherein:
B represents a modified or non-modified purine or pyrimidine base, preferably for adenine, cytosine, guanine, or uracil;
X stands for O or S, preferably O;
Y stands for O or N—CH₃, preferably O; and
Z stands for alkyl, O-alkyl, S-alkyl, NH₂, NH(alkyl), NH(O-alkyl), N(alkyl)₂, N(alkyl)(O-alkyl), preferably N(Alkyl)₂, whereby alkyl stands for linear or branched alkyl groups with 1 to 6 preferably 1 to 3 and especially preferred 1 or 2 carbon atoms. Formulas 1 and 2 only represent an extract from one oligoribonucleotide chain.

Most preferred are morpholine oligoribonucleotides in which the morpholine residues are linked to each other via phosphoryl groups, as shown in Formula 2, in which X stands for O, Y for O and Z for N(CH₃)₂.

Furthermore, ribose residues can also be modified by amino residues, such as NH₂, fluorine, alkyl or O-alkyl residues, such as OCH_{S}, whereby 2′-modified oligoribonucleotides are particularly preferred. Typical modifications are 2′-fluoro, 2′-alkyl, 2′-O-alkyl, 2′-O-methoxyethyl modifications, 5′-palmitate derivates and 2′-O-methylribonucleotides.

Modification of the dsRNA nucleotides counteracts an activation of the protein kinase PKR in the cell, which is dependent on double-stranded RNA. An unspecific inhibition of the translation is prevented by this. The substitution of at least one 2′-hydroxyl group of the dsRNA nucleotides by a 2′-amino or a 2′-methyl group is particularly suitable for this purpose. Moreover, at least one nucleotide in at least one strand of the dsRNA can be replaced by a so-called “locked nucleotide” which contains a chemically modified sugar ring. A preferred modification of the sugar ring is a 2′-O, 4′-C methylene bridge. dsRNA which contains several “locked nucleotides” is preferable.

If not otherwise stated, alkyl in this preferably refers to linear, branched or cyclic alkyl groups with 1 to 30, preferably 1 to 20, especially preferred 1 to 10 and most preferred 1 to 6 carbon atoms. Branched and cyclic residues by their nature exhibit at least 3 carbon atoms, whereby cyclic residues with at least 5 and, in particular, at least 6 carbon atoms are preferred.

Suitable base modifications are described, for example, in U.S. Pat. No. 6,187,578 and WO 99/53101, to which express reference will be made here. A modification of one or more pyrimidines in position 5 with 1, Br, C₁, NH₃ and N₃ has proven to be advantageous.

The synthesis of modified and non-modified oligoribonucleotides as well as further suitable modification options are described in the relevant scientific literature. Moreover, the production of modified and non-modified oligoribonucleotides has meanwhile also been provided as a service by numerous companies, for example Dharmacon, 1376 Miners Drive#101, Lafayette, Colo. 80026, USA, Xeragon Inc., Genset Oligos and Ambion. The production of oligoribonucleotides is also described in U.S. Pat. No. 5,986,084.

Oligoribonucleotides can also be used in encapsulated form to increase the stability and/or penetration, for example encapsulated in liposomes. They can also be stabilized by the addition of cyclodextrines.

Cyclodextrins are also referred to as cycloamyloses and cycloglucans. The cyclodextrins represent cyclic oligosaccharides consisting of a-1,4 concatenated glucose building blocks. Six to eight glucose building blocks (a-, β-, or y-cyclodextrin) are generally linked to each other. Cyclodextrins are obtained for strength under the effect of Bacillus macerans. They possess a hydrophobic interior and a hydrophilic exterior. In accordance with the invention, both the cyclodextrins themselves, in particular a-cyclodextrin, β-cyclodextrin and y-cyclodextrin, as well as the dderivatives thereof are suitable.

The cyclodextrin or cyclodextrins in cosmetic or dermatological compositions are preferably used in a concentration of 0.0005 to 20.0 weight %, in particular 0.01 to 10 weight % and especially preferred in a concentration of 0.1 to 5.0 weight %.

It is advantageous in accordance with the invention to use native, polar and/or apolar substituted cyclodextrins. These preferably include, however not exclusively, methyl-, in particular random-methyl-β-cyclodextrin, ethyl- as well as hydroxypropyl cyclodextrins, for example hydroxypropyl-β-cyclodextrin and hydroxypropyl-y-cyclodextrin. The cyclodextrin species especially preferred in accordance with the invention are y-clodextrin and hydroxypropyl-β-cylcodextrin.

Liposomes can be produced in a familiar manner using natural phospholipids, such as phosphatidylcholine from eggs, soya beans etc., or synthetic phospholipids (cf. G. Betageri (Editor), “Liposome Drug Delivery Systems”, Lancaster Techonomic Publishing Company 1993; Gregoriadis (Editor), “Liposome Technology”, CRC Press). The preferred processes and materials for the production of liposomes are described in WO 99/24018.

Double-stranded oligoribonucleotides can also be modified in order to counteract a dissociation into single strands, for example through one or more covalent, coordinative or ionic bonds. Oligoribonucleotides without such modifications are preferred however.

The nucleotides in the RNA molecules can also comprise “non-standard” nudeotides such as naturally occurring nucleotides or desoxyribonucleotides.

Preferred in accordance with the invention are those such oligoribonucleotides which inhibit the expression of the relevant target gene in comparison to untreated cells by at least 30%, preferably by at least 50%, especially preferred by at least 80% and most preferred by at least 85%. If necessary, the expression of the target gene in the cells is first induced in a suitable manner for measuring the inhibition. Tumoral cells of the line HeLaS3 are preferably used for determining the effectiveness of the oligoribonucleotides according to the invention. The oligoribonucleotides are introduced to the cells and (if necessary after induction of the target gene expression in these cells) the expression rate of the target gene is then measured and compared with the rate which was ascertained in cells which were not transfected with the relevant oligoribonucleotide. The precise conditions for measuring the inhibition can be found in Example 1.

The oligoribonucleotides according to the invention and their salts are particularly suitable as an effective component of pharmaceutical and cosmetic compositions, in particular for topical administration.

Surprisingly, it has turned out that, after application of the compositions to the skin, the oligoribonucleotides inhibit expression of the genes which are responsible for the inflammatory processes of the skin, and thus prevent the formation and distribution of inflammation mediators without side effects and, in this way, enable an effective treatment and prophylaxis of irritations and inflammations of the skin, without manifesting the drawbacks of the prior art. It is assumed that this effect is achieved through a mechanism in which the oligoribonucleotides according to the invention are absorbed by the cells of the skin and induce intracellular degradation of the mRNAs of the relevant genes through RNAi, whereby individual details concerning the mechanism of this reaction cascade are still not known. The oligoribonucleotides are therefore particularly suitable for initiating the degradation of mRNA for structures involved in the inflammation of the skin and for inhibiting such structures in the skin and, in particular, in skin cells.

The pharmaceutical or cosmetic compositions corresponding to the invention preferably contain 0.00001 to 10 weight %, especially preferred 0.0003 to 3 weight % and most preferred 0.01 to 1.0 of the oligoribonucleotide or oligoribonucleotides according to the invention, in relation to the total weight of the composition. When using oligoribonucleotides which are integrated in vectors, the above data concerning amounts refers to the mass of the oligoribonucleotides integrated in the vector, the mass of the vector itself not being considered.

In accordance with the invention, such compositions are preferred which only contain those such oligoribonucleotides which inhibit expression of one or more of the above genes, i.e. the genes of the structures involved in the inflammation and, in particular, the preferred genes cited. The compositions according to the invention can contain one or preferably several oligoribonucleotides. These represent oligoribonucleotides which inhibit the expression of several different structures involved in the inflammation/irritation of the skin.

Mixtures of oligoribonucleotides can also be used, which aim for different sequence regions of one and the same gene or the same mRNA of a structure involved in the skin irritation. Preferred are compositions which contain 1 to 5 and, in particular, 1 to 3 different oligoribonucleotides. Mixtures of oligoribonucleotides which, in addition to the above structures involved in the skin irritation, unspecific ally inhibit or induce the activity of a large number of other skin proteins are undesirable, as practically no control of side effects is possible. The term skin proteins refers to those such proteins which are expressed in the skin. Most preferred are compositions which contain one or more oligoribonucleotides which inhibit the expression of cyclooxygenase or lipoxygenase.

Particularly preferred also are further compositions which each contain at least one oligoribonucleotide which is directed against COX-2.

Also preferred are compositions which each contain at least one oligoribonucleotide which is directed against LOX-5 and/or FLAP.

In addition to this, especially preferred are compositions which each contain at least one oligoribonucleotide which is directed against IL-1a and/or β and/or IL-6 and/or IL-8 and/or TNF-a.

The oligoribonucleotides and compositions are suitable for the treatment and prophylaxis of undesired inflammatory reactions of the skin, in particular of the symptoms described above. They are suitable for the cosmetic and therapeutic treatment of undesired skin irritations which are caused by endogenous and exogenous factors, in particular UV radiation or shaving. The compositions according to the invention can prevent skin irritations and lastingly alleviate existing irritations without the risk of side effects. The process described in WO 02/053773, for example, can be used for determining the effectiveness of the oligoribonucleotides corresponding to the invention.

The oligoribonucleotides according to the invention are particularly suitable for the prevention and treatment of skin irritations caused by sunburn or shaving.

The oligoribonucleotides according to the invention are suitable in an equally preferred form for the prevention and treatment of irritative skin conditions as caused by exogenous stress (e.g. UV radiation, mechanical stress, chemicals). The oligoribonucleotides according to the invention are also suitable for the soothing of sensitive skin types prone to irritations.

The oligoribonucleotides and compositions according to the invention are also tremendously suitable for skin care on the basis of their prophylactic effect.

The compositions according to the invention are also suitable for the treatment of skin damage caused by UV radiation, e.g. the ultraviolet range of sunlight. UV-B radiation (290 to 320 nm) causes, for example, erythemas, sunburn or even more or less intense burns. UV-A radiation (320 nm to 400 nm) can cause irritations in light-sensitive skin and lead to damage to the elastic and collagenous fibers of the connective tissue, which can cause the skin to age prematurely. They are also the cause of numerous phototoxic and photoallergic reactions. The oligoribonucleotides according to the invention are also suitable for the treatment, for example, of structural damage and functional disorders in the epidermis and dermis of the skin caused by UV radiation, for example of visible vascular dilatations such as telangiectasias and cuperosis, skin flabbiness and the formation of wrinkles, local hyper, hypo and defective pigmentation, e.g. age marks, and increased proneness to mechanical stress, e.g. skin chapping.

Further fields of application for the compositions according to the invention are the treatment and prevention of collagen degeneration induced by aging and/or UV radiation as well as the degradation of elastin and glycosaminoglycans; of degenerative symptoms of the skin, such as loss of elasticity as well as atrophy of the epidermal and dermal cell layers, the components of the connective tissue, the retinal cone and capillary vessels and/or the skin adnexa; of negative changes to the skin and the skin adnexa caused by environmental factors, such as ultraviolet radiation, smoking, smog, reactive oxygen species, free radicals and similar; of adverse, sensitive or hypoactive skin conditions or adverse, sensitive or hypoactive conditions of the skin adnexa; of reduction of the skin thickness; of skin limpness and/or skin tiredness; of changes to the transepidermal water loss and the normal moisture content of the skin; of changes to the energy metabolism of healthy skin; of deviations from the normal cell-to-cell communication in the skin which can be expressed by the formation of wrinkles; of changes to the normal fibroblast and keratinocyte proliferation; of changes to the normal fibroblast and keratinocyte differentiation; of polymorphous photodermatosis, vitiligo; of wound healing disorders; of disorders in the normal collagen, hyaluronic acid, elastin and glycosaminoglycan homeostasis; of increased activation of proteolytic enzymes in the skin, e.g. of metalloproteinases.

Compositions for topical administration are preferred in accordance with the invention. The compositions can be extant in all galenical forms which are normally used for a topical administration, such as a solution, cream, ointment, lotion, shampoo, in other words an emulsion of the type water-in-oil (W/O) or of the type oil-in-water (O/W), multiple emulsions, for example of the type water-in-oil-in-water (W/O/W), or oil-in-water-in-oil (O/W), a hydrodispersion or lipodispersion, Pickering emulsion, gel, solid pencil or aerosol.

The cosmetic or medical treatment of the above indications is generally performed through a single or repeated application of the compositions according to the invention to the skin, preferably to the skin areas affected.

The compositions according to the invention are suitable for cosmetic and therapeutic, i.e. especially dermatological, application. The term cosmetic skin care primarily refers to the strengthening or restoration of the natural function of the skin as a barrier against environmental influences (e.g. dirt, chemicals, microorganisms) and against the loss of the body's own substances (e.g. water, natural fats, electrolytes). If this function is damaged, the result can be increased resorption of toxic or allergic substances or attack by microorganisms and consequently toxic or allergic skin reactions.

The objective of skin care is also to compensate the fat and water loss of the skin resulting from daily washing. This is especially important if the natural regeneration ability is not sufficient. Skin care products should also provide protection against environmental influences, in particular sun and wind.

For cosmetic application, the compositions according to the invention therefore preferably contain those such components which are suitable for the above purposes. Such substances are familiar to the expert. For example, one or more anti-sense oligoribonucleotides can be incorporated in standard cosmetic and dermatological preparations, which can be extant in various forms.

In accordance with the especially preferred embodiment, the compositions according to the invention for cosmetic application are extant as an emulsion, e.g. in the form of a cream, lotion, or cosmetic milk. In addition to the oligoribonucleotides cited, these contain further components such as fats, oils, waxes and/or other bodies of fat, as well as water and one or more emulsifiers, as are typically used for such a type of formulation.

Emulsions generally contain a lipid or oil phase, an aqueous phase and preferably also one or more emulsifiers. Particularly preferred are compositions which also contain one or more hydrocolloids.

The compositions according to the invention preferably contain 0.001 to 35 weight %, especially preferred 2 to 15 weight % emulsifier, 0.001 to 45 weight %, especially preferred 10 to 25 weight % lipid, and 10 to 95 weight %, especially preferred 60 to 90 weight % water.

The lipid phase of the cosmetic or dermatological emulsions according to the invention can advantageously be chosen from the following substance group: (1) Mineral oils, mineral waxes; (2) oils such as triglycerides of capric or caprylic acid, also natural oils such as castor oil; (3) Fats, waxes and other natural and synthetic bodies of fat, preferably ester of fatty acids with alcohols of a low C-number, e.g. with isopropanol, propylene glycol or glycerol, or ester of fatty alcohols with alkanoic acids of a low C-number or with fatty acids; (4) Alkyl benzoate; (5) Silicone oils such as dimethylpolysiloxanes, diethylpolysiloxanes, diphenylpolysiloxanes as well as mixed forms of these.

Unless otherwise indicated, the term low C-number preferably refers to 1 to 5, especially preferred 1 to 3 and most preferred 3 carbon atoms here.

The oil phase of the emulsions of the present invention is advantageously chosen from the group of esters from saturated and/or unsaturated, branched and/or unbranched alkane carboxylic acids with a chain length of 3 to 30 carbon atoms and saturated and/or unsaturated, branched and/or unbranched alcohols with a chain length of 3 to 30 carbon atoms, from the group of esters from aromatic carboxylic acids and saturated and/or unsaturated, branched and/or unbranched alcohols with a chain length of 3 to 30 carbon atoms. Such ester oils can then advantageously be chosen from the group isopropyl myristate, isopropyl palmitate, isopropyl stearate isopropyl oleate n-Butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate as well as synthetic, semi-synthetic and natural mixtures of such esters, e.g. jojoba oil.

The oil phase can also advantageously be chosen from the group of branched and unbranched hydrocarbons and waxes, the silicone oils, the dialkyl ethers, the group of saturated or unsaturated, branched or unbranched alcohols, as well as the fatty acid triglycerides, namely the triglycerin ester of saturated and/or unsaturated, branched and/or unbranched alkane carboxylic acids with a chain length of 8 to 24, in particular 12 to 18 carbon atoms. The fatty acid triglycerides can, for example, advantageously be chosen from the group of synthetic, semi-synthetic and natural oils, e.g. olive oil, sunflower oil, soya oil, peanut oil, rape oil, almond oil, palm oil, coconut oil, palm-kernel oil and additional similar such oils.

Any blends of such oil and wax components can also advantageously be used within the sense of the present invention. If necessary, it can also be advantageous to use waxes, for example cetyl palmitate, as sole lipid components of the oil phase.

The oil phase is advantageously chosen from the group 2-ethylhexyl isostearate, octyldodecanol, isotridecyl isononanoate, isoeicosane, 2-ethylhexyl cocoate, C₁₂₋₁₅ alkyl benzoate, caprylic-capric acid triglycerid, dicaprylyl ether.

Particularly advantageous are mixtures of C₁₂₋₁₅ alkyl benzoate and 2-ethylhexyl isostearate, mixtures of C₁₂₋₁₅ alkyl benzoate and isotridecyl isononanoate as well as mixtures of C₁₂₋₁₅ alkyl benzoate, 2-ethylhexyl isostearate and isotridecyl isononanoate.

Of the hydrocarbons, paraffin oil, squalane and squalene are advantageous in the sense of the present invention.

The oil phase can also advantageously exhibit a content of cyclic or linear silicone oils or completely consist of such oils, whereby it is particularly preferred that, in addition to the silicone oil or the silicone oils, an additional content of other oil phase components is used. Such silicone oils can be present as monomers which are generally characterized by structural elements such as the following:

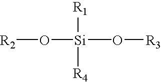

Linear silicones with several siloxyl units to be used advantageously in accordance with the invention are generally characterized by structural elements as follows: 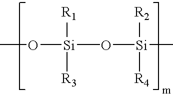
whereby the silicon atoms can be substituted with the same or different alkyl residues and/or aryl residues, which are shown here in general terms by the residues R₁-R₄ (in other words, the number of different residues is not necessarily restricted to 4). For this, m can assume values from 2-200,000. Aryl preferably represents phenyl here, unless otherwise indicated.

Cyclic silicones to be used advantageously in accordance with the invention are generally characterized by structural elements as follows: 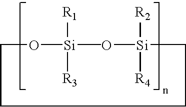
whereby the silicon atoms can be substituted with the same or different alkyl residues and/or aryl residues, which are shown here in general terms by the residues R₁-R₄ (in other words, the number of different residues is not necessarily restricted to 4). For this, n can assume values from 3/2 to 20. Broken values for n allow for the fact that non-linear numbers of siloxyl groups can be present in the cycle.

Cyclomethicone (e.g. decamethylcyclopentasiloxane) is advantageously used as an applicable silicone oil in accordance with the invention. However, other silicone oils can advantageously be used fore the purpose of the present invention, for example undecamethylcyclotrisiloxane, polydimethylsiloxane, poly(methylphenylsiloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated or polypropoxylated. These include, for example, polysiloxane-polyalkyl-polyether copolymers, such as cetyl-dimethicone copolyol, (cetyl-dimethicone copolyol (and) polyglyceryl-4 isostearate (and) hexyl laurate). Also particularly advantageous are mixtures of cyclomethicone and isotridecyl isononanoate, and of cyclomethicone and 2-ethylhexyl isostearate.

If necessary, the aqueous phase of the preparations according to the invention advantageously contains alcohols, diols or polyols having a low C-number, as well as their ethers, preferably ethanol, isopropanol, propylene glycol, glycerol, ethylene glycol, ethylene glycolmonoethyl or -monobutyl ether, propylene glycolmonomethyl, -monoethyl- or -monobutyl ether, diethylene glycolmonomethyl- or -monoethylether and analogous products, as well as alcohols having a low C-number, e.g. ethanol, isopropanol, 1,2-propanediol, glycerol as well as, in particular, one or more thickening agents which can be advantageously chosen from the group silicon dioxide, aluminum silicates.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include (1) partial fatty acid esters and fatty acid esters of polyhydric alcohols and ethoxylated derivatives thereof (e.g. glyceryl monostearates, sorbitan stearates, glyceryl stearyl citrates, sucrose stearates); (2) ethoxylated fatty alcohols and fatty acids; (3) ethoxylated fatty amines, fatty acid amides, fatty acid alkanolamides; (4) alkylphenol polyglycol ethers (e.g. Triton X).

The anionic emulsifiers include soaps (e.g. sodium stearate); fatty alcohol sulfates; mono-, di- and trialkyl phosphoic esters and ethoxylates thereof. The cationic emulsifiers include quaternary ammonium compounds with a long-chain aliphatic radical, e.g. distearyidimonium chloride.

The amphoteric emulsifiers include alkylamininoalkanecarboxylic acids, betaines, sulfobetaines, imidazoline derivates.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g. fatty alcohol ethoxylates, ethoxylated wool wax alcohols, polyethylene glycol ethers of the general formula R—O—(—CH2-CH2-O—)n-R′, fatty acid ethoxylates of the general formula R—COO—(—CH₂—CH₂—O—)ₙ—H, etherified fatty acid ethoxylates of the general formula R—COO—(—CH₂—CH₂—O)ₙ—R′, esterified fatty acid ethoxylates of the general formula R—COO—(—CH₂—CH₂—O—)ₙ—C(O)—R′, polyethylene glycol glycerol fatty acid esters, ethoxylated sorbitan esters, cholesterol ethoxylates, ethoxylated triglycerides, alkyl ether carboxylic acids of the general formula R—O—(CH₂—CH₂—O—)ₙ—CH₂—OOOH, polyoxyethylene sorbitol fatty acid esters, alkyl ether sulfates of the general formula R—O—(—CH₂—CH₂—O—)ₙ—SO₃—H, fatty alcohol propoxylates of the general formula R—O—(—CH₂—CH(CH₃)—O—)ₙ—H, polypropylene glycol ethers of the general formula R—O—(—CH₂—CH(CH₃)—O—)n-R′, propoxylated wool wax alcohols, etherified fatty acid propoxylates, R—COO—(—CH₂—CH(CH₃)—O—)ₙ—R′, esterified fatty acid propoxylates of the general formula R—COO—(—CH₂—CH(CH₃)—O—)ₙ—C(O)—R′, fatty acid propoxylates of the general formula R—COO—(—CH₂—CH(CH₃)—O—)ₙ—H, polypropylene glycol glycerol fatty acid esters, propoxylated sorbitan esters, cholesterol propoxylates, propoxylated triglycerides, alkyl ether carboxylic acids of the general formula R—O—(—CH₂—CH(CH₃)O)ₙ—CH₂—OOOH, alkyl ether sulfates or the parent acids of these sulfates of the general formula R—O—(—CH₂—CH(CH₃)—O—)ₙ—SO₃—H, fatty alcohol ethoxylates/propoxylates of the general formula R—O—XₙYₘ—H, polypropylene glycol ethers of the general formula R—O—XₙYₘ—R′, etherified fatty acid propoxylates of the general formula R—COO—XₙYₘ—R′, fatty acid ethoxylates/propoxylates of the general formula R—COO—XₙYₘ—H.

The variables n and m represent in all cases a whole number from 1 to 40, preferably 5 to 30, independently of each other.

According to the invention, particularly advantageous polyethoxylated or polypropoxylated or polyethoxylated and polypropoxylated O/w emulsifiers used are those chosen from the group of substances having HLB values of 11-18, very particularly preferably having HLB values of 14.5-15.5, provided the ONV emulsifiers have saturated radicals R and R′. If the O/N emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to: polyethylene glycol(13) stearyl ether (steareth-13), polyethylene glycol(14) stearyl ether (steareth-14), polyethylene glycol(15) stearyl 10 ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group: polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate. The ethoxylated alkyl ether carboxylic acid or salt thereof which can be used is advantageously sodium laureth-1 1-carboxylate.

Sodium laureth 1-4-sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) evening primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated or unsaturated, branched or unbranched alkanecarboxylic acids having a chain length of from 8 to 24, in particular 12-18, carbon atoms, monoglycerol ethers of saturated or unsaturated, branched or unbranched alcohols having a chain length of from 8 to 24, in particular 12-18, carbon atoms, diglycerol ethers of saturated or unsaturated, branched or unbranched alcohols having a chain length of from 8 to 24, in particular 12-18, carbon atoms, propylene glycol esters of saturated or unsaturated, branched or unbranched alkanecarboxylic acids having a chain length of from 8 to 24, in particular 12-18, carbon atoms, and sorbitan esters of saturated or unsaturated, branched or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate, glyceryl monocaprylate.

Preparations according to the invention in the form of emulsions also advantageously comprise one or more hydrocolloids. These hydrocolloids can advantageously be chosen from the group of gums, polysaccharides, cellulose derivatives, phyllosilicates, polyacrylates and/or other polymers.

Preparations according to the invention in the form of hydrogels also advantageously comprise one or more hydrocolloids. These hydrocolloids can advantageously be chosen from the above group.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar® HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorite.

In addition, silica gels can also be used advantageously.

The polyacrylates include, for example, Carbopol grades from Goodrich (Carbopol 980, 981, 1382, 5984, 2984, EDT 2001 or Pemulen TR2).

The polymers include, for example, polyacrylamides (Seppigel 305), polyvinyl alcohols, PVP, PVP/VA copolymers, polyglycols.

According to a further preferred embodiment, the oligoribonucleotides used are added to aqueous systems or surfactant preparations for cleaning the skin and hair.

In addition to the components cited, the cosmetic preparations according to the invention also preferably contain auxiliary agents, as are typically used in such preparations, e.g. preservatives, bactericides, substances with a deodorant effect, anti-perspirants, insect repellants, vitamins, anti-foaming agents, colorings, pigments with coloring effect, thickening agents, softening agents, moisturizing and/or moisture-retaining substances (moisturizers), or other standard components of a cosmetic formulation such as polyols, polymers, foam stabilizers, electrolytes, organic solvents or silicone derivatives, antioxidants and, in particular, UV absorbers.

Designated as moisturizers are substances and mixtures of substances which lend cosmetic or dermatological preparations the property, after application or spreading on the skin surface, of reducing the moisture loss of the stratum corneum (also referred to as transepidermal water loss (TEWL)) and/or positively influencing the hydratation of the stratum corneum.

Advantageous moisturizers for the purpose of the present invention are, for example, glycerol, lactic acid, pyroglutamic acid and urea. It is also of particular advantage to use polymeric moisturizers from the group of water-soluble polysaccharides and/or polysaccharides swellable in water and/or polysaccharides which can be gelatinized with the aid of water. Particularly advantageous, for example, is hyaluronic acid and/or a fucose-rich polysaccharide which is recorded in the Chemical Abstracts under the registration number 178463-23-5 and which is typically available from the company SOLABIA S.A. under the designation Fucogel®1000.

When used as a moisturizer, glycerol is preferably used in a quantity of 0.05-30 weight %, particularly preferred is 1-10%.

The cosmetic compositions can also advantageously contain one or more of the following natural active substances or a derivative thereof: alpha-lipoc acid, phytoene, D-biotin, coenzyme Q10, alpha glucosylrutin, carnitine, carnosine, natural and/or synthetic isoflavonoides, creatine, hops or hops-malt extract, taurine. It turned out that active substances with a positive effect on aging skin, which reduce the formation of wrinkles or lessen existing wrinkles, such as biquinones and, in particular, ubiquinone Q10, soya, creatinine, creatine, liponamide, or aid the restructuring of connective tissue, such as isoflavone, can be used highly effectively in the formulations according to the invention. It was also revealed that the formulations are especially suitable for combination with active substances for supporting the skin functions with dry skin, in particular dry skin due to aging, such as serinol and osmolytes, e.g. taurine. The incorporation of modulators for pigmentation also proved to be advantageous. These include active substances which reduce pigmentation of the skin and hence achieve a cosmetically desirable brightening of the skin and/or reduce the occurrence of age marks and/or brighten existing age marks (tyrosine sulfate, dioic acid (8-hexadecen-1,16-dicarbon acid), lipoic acid and liponamide, various extracts of liquorice, kojic acid, hydroquinone, arbutin, fruit acids, in particular alpha-hydroxy acids (AHAs), bearberry (Uvae ursi), ursolic acid, ascorbic acid, green tea extracts).

Corresponding to a particularly preferred embodiment, the compositions according to the invention contain one or more UV absorbers. Preferred UV absorbers are those which absorb in the range of UV-B and UB-A radiation.

Numerous compounds involving derivatives of 3-benzylidene camphor, 4-aminobenzoic acid, cinnamic acid, salicylic acid, benzophenone as well as 2-phenylbenzimidazole are familiar as protection against UV-B radiation. Preferable are filters with an absorption maximum in the range of 308 nm, as the maximum erythema-inducing effect of sunlight lies within this range.

Advantageous UV-A filter substances for the purpose of the present invention are dibenzoyl methane derivates, in particular 4-(tert.-Butyl)-4′-methoxydibenzoylmethane (CAS No. 70356-09-1), which is sold by Givaudan under the brand name Parsol® 1789 and by Merck under the trade name Eusolex® 9020.

The preparations according to the invention contain advantageous substances which absorb UV radiation in the UV-A and/or UV-B range, whereby the total amount of filter substances is e.g. 0.1 weight % to 30 weight %, preferably 0.5 to 20 weight %, in particular 1.0 to 15.0 weight %, in relation to the total weight of the preparations, in order to provide cosmetic preparations which protect the skin or hair against the entire spectrum of ultraviolet radiation. They can also serve as sunscreens for the hair or skin.

Further advantageous UV-A filter substances are phenylene-1,4-bis-(2-benzimidazyl)-3,3′-5,5′-tetrasulfonic acid 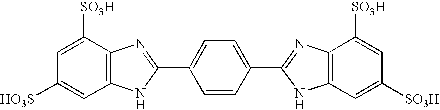
and its salts, especially the corresponding sodium, potassium or triethanolammonium salts, in particular phenylene-1,4-bis-(2-benzimidazyl)-3,3′-5,5′-tetrasulfonic acid bis-sodium salt 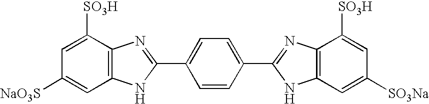
with the INCI name Bisimidazylate, which is available, for example, under the trade name Neo Heliopan AP from Haarmann & Reimer.

Also advantageous is 1,4-di(2-oxo-10-sulfo-3-bornylidenemethyl)benzene and salts thereof (preferably the corresponding 10-sulfato compounds, in particular the corresponding sodium, potassium or triethanolammonium salt), which is also referred to as benzene-1,4-di(2-oxo-3-bornylidenemethyl-10-sulfonic acid), and which is characterized by the following formula:

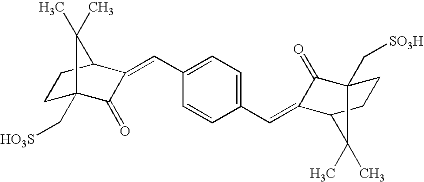

Advantageous UV filter substances for the purposes of the present invention are also so-called broadband filters, i.e. filter substances which absorb both UV-A and UV-B radiation.

Advantageous broadband filters or UV-B filter substances are, for example, bis-resorcinyltriazine derivates with the following structure: 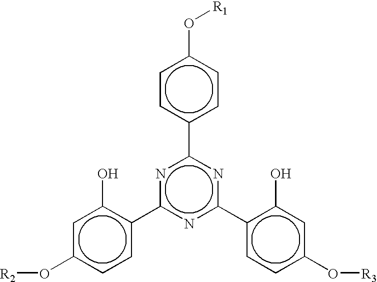
whereby R′, R² and R³ are chosen independently of each other from the group of branched and unbranched alkyl groups with 1 to 10 carbon atoms or represent a single carbon atom. Particular preference is given to 2,4-bis{[4-(2-ethylhexyl-oxy)-2-hydroxy]phenyl}-6-(4-methoxyphenyl)-1,3,5-triazine (INCI: Bisethylhexyloxyphenol Methoxyphenyl Triazine), which is available under the trade name Tinosorb® S from CIBA-Chemikalien GmbH, and 4,4′,4″-(1,3,5-triazine-2,4,6-triyltriimino)-tris-benzoic acid tris(2-ethylhexylester), synonym: 2,4,6-tris-[anilino-(p-carbo-2′-ethyl-l′-hexyloxy)]-1,3,5-triazin (INCI: Octyl Triazone), which is available from BASF Aktiengesellschaft under the product name UVINUL® T 150.

Moreover other UV filter substances which exhibit the structure 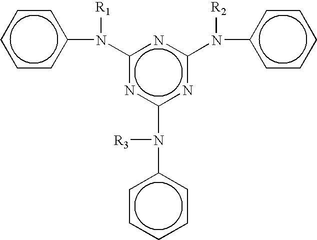
are also advantageous UV filter substances for the purpose of the present invention, for example the s-triazine derivatives described in the European Published Patent Application EP 570 838 A1, whose chemical structure is represented by the generic formula 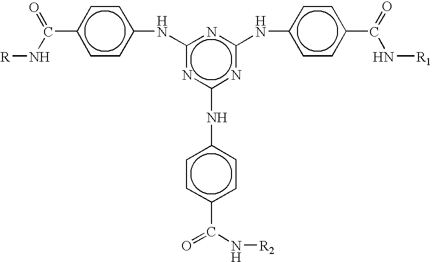
whereby
R represents a branched or unbranched C₁-C₁₈ alkyl residue, a C₅-C₁₂ cycloalkyl residue, if necessary substituted with one or more C₁-C₄ alkyl groups,
X represents an oxygen atom or an NH group,
R₁ represents a branched or unbranched C₁-C₁₈ alkyl residue, a C₅-C₁₂ cycloalkyl residue, if necessary substituted with one or more C₁-C₄ alkyl groups, or a hydrogen atom, an alkali metal atom, an ammonium group, or a group having the formula: 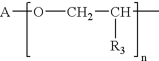
   in which
A represents a branched or unbranched C₁-C₁₈ alkyl residue, a C₅-C₁₂ cycloalkyl or aryl residue, if necessary substituted with one or more C₁-C₄ alkyl groups,
R₃ represents a hydrogen atom or a methyl group,
n represents a number from 1 to 10,
if X represents an oxygen atom.

An especially advantageous UV filter substance for the purpose of the present invention is also an asymmetrically substituted s-triazine whose chemical structure is represented by the formula 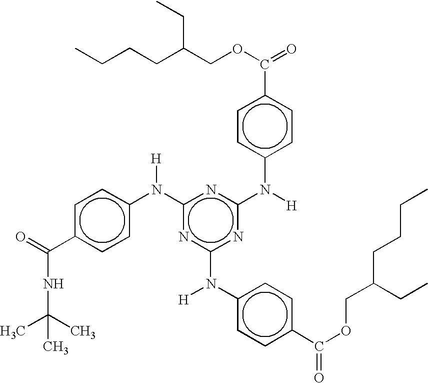
which is referred to in the following as dioctylbutylamidotriazone (INCI: Dioctylbutamidotriazone) and which is available from Sigma 3V under the trade name UVA-SORB HEB.

Also described in European Printed Patent Application EP 775 698 are bis-resorcinyltriazine derivatives which can be used advantageously and whose chemical structure is represented by the generic formula 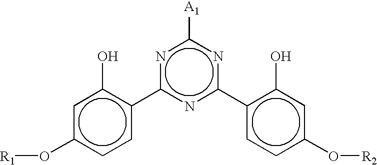
whereby R₁, R₂ and A₁ represent highly diverse organic residues.

Advantageous for the purpose of the present invention is also 2,4-Bis-{[4-(3-sulfonato)-2-hydroxy-propyloxy)-2-hydroxy]-phenyl}-6-(4-methoxyphenyl)-1,3,5-triazin sodium salt, 2,4-Bis-{[4-(3-(2-Propyloxy)-2-hydroxy-propyloxy)-2-hydroxy]-phenyl}-6-(4-methoxy-phenyl)-1,3,5-triazin, 2,4-Bis-{[4-(2-ethyl-hexyloxy)-2-hydroxy]-phenyl}-6-[4-(2-meth-oxyethyl-carboxyl)-phenylamino]-1,3,5-triazin, 2,4-Bis-{[4-(3-(2-propyloxy)-2-hydroxy-propyloxy)-2-hydroxy]-phenyl}-6-[4-(2-ethyl-carboxyl)-phenylamino]-1,3,5-triazin, 2,4-Bis-{[4-(2-ethyl-hexyloxy)-2-hydroxy]-phenyl}-6-(1-methyl-pyrrol-2-yl)-1,3,5-triazin, 2,4-Bis-{[4-tris(tri methylsiloxy-silyl propyloxy)-2-hydroxy]-phenyl}-6-(4-methoxyphenyl)-1,3,5-triazin, 2,4-Bis-{[4-(2″-methylpropenyloxy)-2-hydroxy]-phenyl}-6-(4-methoxyphenyl)-1,3,5-triazin and 2,4-Bis-{[4-(1′,1′,1′,3′,5′,5′,5′-heptamethylsiloxy-2″-methylpropyloxy)-2-hydroxy]-phenyl-6-(4-methoxyphenyl)-1,3,5-triazin.

An advantageous broadband filter for the purpose of the present invention is 2,2′-methylene-bis-(6-(2H-benzotriazol-2-yl)-4-(1,1,3,3-tetramethylbutyl)-phenol) [INCI: Bisoctyltriazol], which is characterized by the chemical structural formula 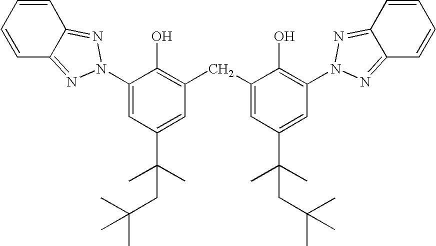
and which is available under the trade name Tinosorb® M from CIBA-Chemikalien GmbH.

An advantageous broadband filter for the purpose of the present invention is also 2-(2H-benzotriazol-2-yl)₄-methyl-6-[2-methyl-3-[1,3,3,3-tetramethyl-1-[(trimethylsilyl)oxy]di-siloxanyl]propyl]-phenol (CAS No.: 155633-54-8) with the INCI designation Drometrizole Trisiloxane, which is characterized by the chemical structural formula

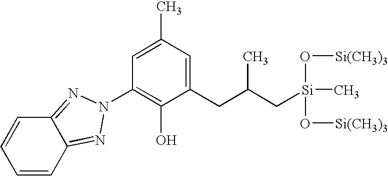

The UV-B filters can be oil-soluble or water-soluble. Advantageous oil-soluble UV-B filters are, for example, 3-benzylidene camphor derivates, preferably 3-(4-methyl-benzylidene) camphor, 3-benzylidene camphor; 4-aminobenzoic acid derivatives, preferably 4-(dimethylamino)-benzoic acid (2-ethylhexyl)ester, 4-(dimethylamino) benzoic acid amylester; 2,4,6-trianilino-(p-carbo-2′-ethyl-l′-hexyloxy)-1,3,5-triazin; ester of benzylmalonic acid, preferably 4-methoxy-benzylmalonic acid di(2-ethylhexyl)ester; ester of cinnamic acid, preferably 4-methoxy cinnamic acid (2-ethylhexyl)ester, 4-methoxy cinnamic acid isopentylester; derivates of benzophenone, preferably 2-hydroxy-4-methoxybenzophenone, 2-hydroxy-4-methoxy-4′-methylbenzophenone, 2,2′-dihydroxy-4-methoxybenzophenone as well as UV filters bonded to polymers.

Advantageous water-soluble UV-B filter substances are, for example, salts of 2-phenylbenzimidazole-5-sulfonic acid, such as sodium, potassium or triethanolammonium salt thereof, as well as sulfonic acid itself; sulfonic acid derivatives of 3-benzylidene camphor, such as 4-(2-oxo-3-bornylidenmethyl)benzene sulfonic acid, 2-methyl-5-(2-oxo-3-bornylidenmethyl)sulfonic acid and salts thereof.

A further light filter substance to be used advantageously in accordance with the invention is ethylhexyl-2-cyano-3,3-diphenylacrylate (octocrylene), which is available from BASF under the name Uvinul® N 539 and which is characterized by the following structure:

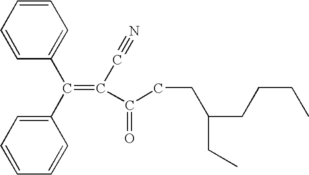

It can also be of considerable advantage to use polymer-bonded or polymeric UV filter substances in preparations according to the present invention, in particular as described in WO-A-92/20690.

It can also be advantageous, if required, to incorporate further UV-A and/or UV-B filters in cosmetic or dermatological preparations according to the invention, for example certain salicylic acid derivates such as 4-isopropylbenzyl salicylate, 2-ethylhexyl salicylate (=octyl salicylate), homomenthyl salicylate.

The list of UV filters cited which can be used for the purpose of the present invention, should not be limiting of course.

The preparations according to the invention can also contain antioxidants for protection of the cosmetic preparation itself or for protection of the components of the cosmetic preparations against harmful oxidation processes.

The antioxidants are advantageously chosen from the group consisting of amino acids (e.g. glycine, histidine, tyrosins, tryptophan) and derivatives thereof, imidazoles (e.g. urocanic acid) and derivates thereof, peptides such as D,L-carnosine, D-carnosine, L-carnosins and derivates thereof (e.g. anserin), carotenoids, carotenes (e.g. a-carotene, β-carotene, lycopene) and derivatives thereof, aurothioglucose, propylthiouracil and other thiols (e.g. thioredoxin, glutathione, cysteine, cystine, cystamine and glycosyl-, N-acetyl-, methyl-, ethyl-, propyl-, amyl-, butyl- and lauryl-, palmitoyl-, oleyl-, y-linoleyl-, cholesteryl- and glyceryl esters thereof) as well as salts thereof, dilauryl thiodipropionate, distearyl thiodipropionate, thiodipropionic acid derivatives thereof (esters, ethers, peptides, lipids, nucleotides, nucleosides and salts) as well as sulfoximine compounds (e.g. buthioninsulfoximines, homocystein sulfoximine, buthionine sulfones, penta-, hexa-, heptathionine sulfoximine) in very low compatible doses (e.g. pmol to pmol/kg), also (metal)-chelators (e.g. a-hydroxy fatty acids, palmitic acid, phytic acid, lactoferrin), a-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. y-linolenic acid, linolic acid, oleic acid), folic acid and derivatives thereof, alanine diacetic acid, flavonoids, polyphenols, catechins, vitamin C and derivatives (e.g. ascorbyl palmitate, Mg-ascorbyl phosphate, ascorbyl acetate), tocopherols and derivatives (e.g. vitamin E acetate), as well as coniferyl benzoate of benzoin resin, rutinic acid and derivatives thereof, ferulic acid and derivatives thereof, butylhydroxytoluol, butylhydroxyanisol, nordihydroguajak resin acid, nordihydroguajaret acid, trihydroxybutyrophenone, uric acid and derivatives thereof, mannose and derivatives thereof, zinc and derivatives thereof (e.g. ZnO, ZnSO₄) selenium and derivatives thereof (e.g. selenmethionine), stilbenes and selenmethionin (e.g. stilbene oxide, trans-stilbene oxide) and the derivatives of these active substances suitable according to the invention (salts, esters, ethers, sugar, nudeotides, nucleosides, peptides and lipids).

The amount of antioxidants (one or more compounds) in the preparations is preferably 0.001 to 30 weight %, especially preferred 0.05-20 weight %, in particular 1-10 weight %, in relation to the total weight of the preparation. Cosmetic and therapeutic preparations according to the invention advantageously contain inorganic pigments based on metal oxides and/or other metal compounds which are slightly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminum (A12O3), cerium (e.g. Ce₂O₃), mixed oxides of the corresponding metals as well as blends of such oxides. Especially preferred are pigments based on TiO₂.

It is particularly advantageous for the purpose of the present invention, if not essential, if the inorganic pigments are present in hydrophobic form, i.e. they have been surface water-repellant treated. This surface treatment can involve these pigments receiving a thin hydrophobic layer according to familiar processes.

One of these such processes, for example, involves creating the hydrophobic surface layer after a reaction according to
nTiO₂+m(RO)₃Si—R′->nTiO₂ (surface)

For this, n and m are any stoichiometric parameters to be used, R and R′ the required organic residues. For example, hydrophobically-treated pigments represented analogous to DE-OS 33 14 742 are advantageous.

Advantageous TiO₂ pigments are typically available from the company TAYCA under the trade name MT 100 T, or as M 160 from the company Kemira as well as T 805 from the company Degussa.

Preparations according to the invention can also contain anionic, non-ionic and/or amphoteric surfactants, especially if crystalline or micro-crystalline solids (for example inorganic micropigments) are to be incorporated in the preparations. Surfactants are amphiphilic substances which can dissolve organic, apolar substances in water.

The hydrophilic parts of a surfactant molecule are generally polar functional groups, for example —COO⁻, —O5O₃²⁻, —SO₃⁻, while the hydrophobic parts generally represent apolar carbohydrates. Surfactants are usually classified according to the type and charge of the hydrophilic molecule part. At the same time, four groups can be distinguished, namely anionic surfactants, cationic surfactants, amphoteric surfactants and non-ionic surfactants.

Anionic surfactants generally exhibit carboxylate, sulfate or sulfonate groups as functional groups. In an aqueous solution they form negatively charged organic ions in an acid or neutral environment. Cationic surfactants are almost exclusively characterized by the presence of a quaternary ammonium group. In an aqueous solution they form positively charged organic ions in an acid or neutral environment. Amphoteric surfactants contain both anionic and cationic groups and thus behave like anionic or cationic surfactants in an aqueous solution, depending on the pH value. They have a positive charge in a highly acid environment and a negative charge in an alkaline environment. However, they are zwitterionic in a neutral pH range, as the following example should make clear:
pH=2 RNH₂⁺CH₂CH₂OOOHX
(X⁻=any anion, e.g. Cl⁻)
pH=7 RNH₂⁺CH₂CH₂OOO
pH=12RNHCH₂CH₂OOO⁻B⁺
(B⁻′=any cation, e.g. Na⁻′)

Polyether chains are typical for non-ionic surfactants. Non-ionic surfactants do not form any ions in an aqueous medium.

Anionic surfactants which can be used advantageously are: Acylamino acids (and salts thereof), such as (1) Acylglutamates, for example sodium acylglutamate, Di-TEA palmitoyl aspartate and sodium caprylic/capric glutamate; (2) Acyl-peptides, for example palmitoyl hydrolyzed milk protein, sodium cocoyl-hydrolyzed soya protein and sodium/potassium cocoyl hydrolyzed collagen; (3) Sarcosinates, for example myristoyl sarcosine, TEA lauroyl sarcosinate, sodium lauroyl sarcosinate and sodium cocoyl sarcosinate; (4) Taurates, for example sodium lauroyl taurate and sodium methyl cocoyl taurate; (5) Acyl lactylates, such as lauroyl lactylate and caproyl lactylate; (6) Alaninates;
Carboxylic acids and derivatives, such as lauric acid, aluminum stearate, magnesium alkanolate and zinc undecylenate; ester carboxylic acids, for example calcium stearoyl lactylate, laureth-6 citrate and sodium PEG-4 lauramid carboxylate; ether carboxylic acids, for example sodium laureth-13 carboxylate and sodium PEG-6 cocamide carboxylate;
Carboxylic acids, ester carboxylic acids and ether carboxylic acids preferably contain 1 to 50 and in particular 2 to 30 carbon atoms.

Phosphoric ester and salts, such as DEA-oleth-10-phosphate and dilaureth-4 phosphate;
Sulfonic acids and salts, such as (1) Acyl isethionates, e.g. sodium/ammonium cocoyl isethionate; (2) Alkylaryl sulfonates; (3) Alkyl sulfonates, for example sodium cocosmonoglyceride sulfate, sodium C₁₂_{_{—}}₁₄ olefin-sulfonate, sodium lauryl sulfoacetate and magnesium PEG-3 cocamide sulfate; (4) Sulfosuccinates, for example dioctyl sodium sulfosuccinate, disodium laureth sulfosuccinate, disodium lauryl sulfosuccinate and disodium undecylenamido-MEA sulfosuccinate;
Sulfuric esters, such as (1) Alkyl ether sulfate, for example sodium, ammonium, magnesium, MIPA, TIPA laureth sulfate, sodium myreth sulfate and sodium C₁₂_{_{—}}₁₃ pareth sulfate; (2) Alkyl sulfates, for example sodium, ammonium and TEA laurylsulfate. Cationic surfactants to be used advantageously are alkylamines, alkylimidazoles, ethoxylated amines and quaternary surfactants as well as esterquats.

Quaternary surfactants contain at least one N atom which is bonded covalently to 4 alkyl or aryl groups. Irrespective of the pH, this leads to a positive charge. Alkylbetaine, alkylamidopropylbetaine and alkylamidopropylhydroxysultaine are advantageous. The cationic surfactants used according to the invention can also be chosen preferably from the group of quaternary ammonium compounds, in particular benzyltrialkyl ammonium chloride or bromide, such as benzyldimethylstearyl ammonium chloride, also alkyltrialkyl ammonium salts, for example cetyltrimethylammonium chloride or -bromide, alkyldimethylhydroxyethylammonium chlorides or -bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds having cationic character, such as amine oxides, for example alkyldimethylamine oxides or alkylaminoethyl-dimethylamine oxides. In particular the use of cetyltrimethylammonium salts is advantageous.

Amphoteric surfactants which can be used advantageously are (1) Acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate; (2) N-alkylamino acids, for example aminopropylalkylglutamide, alkylaminopropionic acid, sodium alkylimidodipropionate and lauroamphocarboxyglycinate.

Nonionic surfactants which can be used advantageously are (1) Alcohols; (2) Alkanolamides, such as cocamides MEA/DEA/MIPA; (3) Amine oxides, such as cocoamidopropylamine oxide; (4) Esters which are formed by esterification of carboxylic acids with ethylene oxide, glycerol, sorbitan or other alcohols; (5) Ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated propoxylated lanolin, ethoxylated/propoxylated polysiloxanes, propoxylated POE ethers and alkyl polyglycosides, such as lauryl glucoside, decyl glycoside and cocoglycoside; (6) Sucrose esters, sucrose ethers; (7) Polyglycerol esters, diglycerol esters, monoglycerol esters; (8) Methylglucose esters, esters of hydroxy acids.

The use of a combination of anionic and/or amphoteric surfactants with one or more non-ionic surfactants is also advantageous.

The surface-active substance can be in a concentration between 1 and 95 weight % in the preparations according to the invention, in relation to the total weight of the preparations.

Preparations for medical application do not differ from the cosmetic products in their composition and can also contain the above substances. They are primarily distinguished from the latter through the fact that they have to pass through a special approval procedure.

The invention is explained in more detail on the basis of the embodiment examples. All numerical data in the examples refers to weight %, if not otherwise indicated.

### EXAMPLES

### PIT Emulsions

### Example 1

### Production of PIT Emulsions

Mixing the components cited in the table produced phase-inversion-temperature emulsions (PIT emulsions) for the composition also cited. dsRNA was used as an oligoribonucleotide, this being obtained through hybridization of the sequences SEQ ID NOs 120 and 135. The dsRNA exhibits continuously on the 3′ two protruding dT residues each. The dsRNA is specific to the cDNA of the cycloxygenase and inhibits expression of the gene for this enzyme through RNA interference. It is therefore designated as anti-cyclooxygenase dsRNA. The other abbreviations used in the examples are to be understood accordingly.

| | Emulsion No. | | | | |
|---|---|---|---|---|---|
| | 1 | 2 | 3 | 4 | 5 |
| Glycerin monostearate self-emulsifying | 0.50 | | 3.00 | 2.00 | 4.00 |
| Polyoxyethylene(12)cetylstearylether | | 5.00 | | 1.00 | 1.50 |
| Polyoxyethylene(20)cetylstearylether | | | | 2.00 | |
| Polyoxyethylene(30)cetylstearylether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis-(4-(2-ethyl-hexyloxy-)2-hydroxyl)- | | 1.50 | | 2.00 | 2.50 |
| phenyl)-6-(4-methoxyphenyl)-(1,3,5)- | | | | | |
| triazine | | | | | |
| 1-(4-tert-Butyl phenyl)-3-(4-methoxyphenyl)- | | | 2.00 | | |
| 1,3-propanedione | | | | | |
| Diethylhexyl Butamidotriazone | 1.00 | 2.00 | | 2.00 | |
| Ethylhexyl Triazone | 4.00 | | 3.00 | 4.00 | |
| 4-Methylbenzylidene camphor | | 4.00 | | | 2.00 |
| Octocrylene | | 4.00 | | | 2.50 |
| Phenylene-1,4-bis-(monosodium, 2 | | | 0.50 | | 1.50 |
| benzimidazyl-5,7-disulfonic acid | | | | | |
| Phenylbenzimidazole sulfonic acid | 0.50 | | | 3.00 | |
| C12-15 Alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea Butter (Sheabutter) | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Anti-cyclooxygenase-dsRNA (dsRNA from | 0.10 | 0.10 | | 0.10 | 0.10 |
| SEQ ID NOs 120 and 135) | | | | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad. 100 | ad. 100 | ad. 100 | ad. 100 | ad. 100 |

A PIT emulsion was produced analogously using dsRNA, which was obtained through hybridization of the sequences SEQ NOs 130 and 131. The indications of quantity for anti-COX-2-dsRNA refer to the total amount of dsRNA which is composed of the respective sequences (SEQ IDs) cited.

### Example 2

### Production of Creams Based on Oil-in-Water Emulsions

Mixing the components cited in the table produced creams of the composition also cited.

O/W Creams

| | Cream No. | | | | |
|---|---|---|---|---|---|
| | 1 | 2 | 3 | 4 | 5 |
| Glyceryl stearate citrate | | | 2.00 | | 2.00 |
| Glyceryl state, self-emulsifying | 4.00 | 3.00 | | | |
| PEG-40 stearate | 1.00 | | | | |
| Polyglyceryl-3-methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetyl stearyl alcohol | | | | | 2.00 |
| Caprylic/Capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidene camphor | | | 1.00 | | |
| 1-(4-tert-Butyl phenyl)-3-(4-methoxyphenyl)- | | | 0.50 | | |
| 1,3-propanedione | | | | | |
| Anti-LOX-5-dsRNA (dsRNA from SEQ ID | 0.10 | 0.10 | 0.10 | 0.10 | 0.10 |
| NOs 95, 97 and 101) | | | | | |
| Anti-Cyclooxygenase-dsRNA (dsRNA from | 0.10 | 0.10 | 0.10 | 0.10 | 0.10 |
| SEQ ID NOs 120, 135 and 182) | | | | | |
| Tocopherol | 0.1 | | | | 0.20 |
| Biotin | | | 0.05 | | |
| Ethylenediaminetetraacetic acid trisodium | 0.1 | | 0.10 | 0.1 | |
| Preservative | q.s. | q.s. | q.s. | q.s. | q.s. |
| Polyacrylic acid | 3.00 | 0.1 | | 0.1 | 0.1 |
| Sodium hydroxide solution 45% | q.s | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | Ad 100 | Ad 100 | Ad 100 | Ad 100 |

| | Cream No. | | | | |
|---|---|---|---|---|---|
| | 6 | 7 | 8 | 9 | 10 |
| Glyceryl stearate citrate | | 2.00 | 2.00 | | |
| Glyceryl state, self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetyl stearyl alcohol | | 3.00 | 1.00 | | 0.50 |
| C12-15 Alkyl benzoate | | 2.00 | 3.00 | | |
| Caprylic/Capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidene camphor | 1.00 | | | | 1.00 |
| 1-(4-tert-Butylphenyl)-3-(4-methoxyphenyl)- | 0.50 | | | | 0.50 |
| 1,3-propanedione | | | | | |
| Anti-COX-2-dsRNA (dsRNA from SEQ ID | 0.10 | 0.10 | 0.10 | 0.10 | 0.10 |
| Nos 122 and 159) | | | | | |
| Anti-IL-6-dsRNA (dsRNA from SEQ ID NOs | | | | | |
| 50, 57 and 55) | | | | | |
| Anti-LOX-5-dsRNA (dsRNA from SEQ ID | | | | | |
| NOs 96, 100 and 103) | | | | | |
| Tocopherol | | | | | 0.05 |
| Ethylenediaminetetraacetic acid trisodium | | | 0.20 | | 0.20 |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | Ad 100 | Ad 100 | Ad 100 | Ad 100 | Ad 100 |

A cream was produced analogously using dsRNA, which was obtained through hybridization of the sequences SEQ NOs 137 and 172. The indications of quantity for anti-COX-2-dsRNA, anti-IL-6-dsRNA and anti-LOX-5-dsRNA refer to the total amount of dsRNA which is composed of the respective sequences (SEQ IDs) cited.

### Example 3

### Production of Water-in-Oil Emulsions

Mixing the components cited in the table produced water-in-oil emulsions of the composition also cited. dsRNA was used as an oligoribonucleotide, this being obtained through hybridization of the sense RNA and anti-sense RNA strand for SEQ ID NO 183. SEQ ID NO 183 is a section of the cDNA of the cyclooxigenase. Both strands of the dsRNA exhibit continuously on the 3′ side two 2′-desoxythymidine residues each. The indications of quantity for anti-COX-2-dsRNA refer to the total amount of dsRNA which is composed of the respective sequences (SEQ IDs) cited.

W/O Emulsions

| | Emulsion No. | | | | |
|---|---|---|---|---|---|
| | 1 | 2 | 3 | 4 | 5 |
| Cetyl dimethicone copolyol | | 2.50 | | 4.00 | |
| Polyglyceryl-2-dipolyhydroxystearate | 5.00 | | | | 4.50 |
| PEG-30 dipolyhydroxystearate | | | 5.00 | | |
| 2-Ethylhexyl methoxy cinnamate | | 8.00 | | 5.00 | 4.00 |
| 2,4-Bis-(4-(2-ethyl-hexyloxy)-2-hydroxyl)- | 2.00 | 2.50 | | 2.00 | 2.50 |
| phenyl)-6-(4-methoxyphenyl)-(1,3,5)-triazine | | | | | |
| 1-(4-tert-Butyl phenyl)-3-(4-methoxyphenyl)- | | | 2.00 | 1.00 | |
| 1,3-propanedione | | | | | |
| Diethylhexyl Butamido triazone | 3.00 | 1.00 | | | 3.00 |
| Ethylhexyl triazone | | | 3.00 | 4.00 | |
| 4-Methylbenzylidene Camphor | | 2.00 | | 4.00 | 2.00 |
| Octocrylene | 7.00 | 2.50 | 4.00 | | 2.50 |
| Diethylhexyl Butamido triazone | 1.00 | | | 2.00 | |
| Phenylene-1,4-bis-(monosodium,2- | 1.00 | 2.00 | 0.50 | | |
| benzimidazyl-5,7-disulfonic acid) | | | | | |
| Phenylbenzimidazole sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| C12-15 Alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.0 | | | 2.00 |
| Shea Butter | | | 3.00 | | |
| PVP hexadecene copolymer | 0.50 | | | 0.50 | 1.00 |
| Octoxyglycerin | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Anti-COX-2-dsRNA (dsRNA from SEQ ID | 0.10 | 0.10 | 0.10 | 0.10 | 0.10 |
| No 160, 163 and 178) | | | | | |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad. 100 | ad. 100 | ad. 100 | ad. 100 | ad. 100 |

Emulsion No.

| | 6 | 7 |
|---|---|---|
| Cetyl dimethicone copolyol | | |
| Polyglyceryl-2-dipolyhydroxystearate | 4.00 | 5.00 |
| PEG-30 dipolyhydroxystearate | 0.50 | 1.50 |
| Lanolin alcohol | | |
| Isohexadecane | 1.00 | 2.00 |
| Myristyl myristate | 0.50 | 1.50 |
| Vaseline | 1.00 | 2.00 |
| 1-(4-tert-Butylphenyl)-3- | 0.50 | 1.50 |
| (4-methoxyphenyl)-1,3-propanedione | | |
| 4-Methylbenzylidene camphor | 1.00 | 3.00 |
| Butylene glycol dicaprylate/dicaprate | 4.00 | 5.00 |
| Shea Butter | | 0.50 |
| Butylene glycol | | 6.00 |
| Octoxyglycerin | | 3.00 |
| Glycerol | 5.00 | |

Hydrodispersions

| | | |
|---|---|---|
| Tocopherol acetate | 0.50 | 1.00 |
| Anti-FLAP-dsRNA (dsRNA from | 0.10 | 0.10 |
| SEQ ID Nos 105 and 118) | | |
| Trisodium EDTA | 0.20 | 0.20 |
| Preservative | q.s. | q.s. |
| Ethanol | | 3.00 |
| Perfume | q.s. | q.s. |
| Water | ad. 100 | ad. 100 |

### Example 4

### Production of Hydrodispersions

Mixing the components cited in the table produced hydrodispersions of the composition also cited. dsRNA was used as an oligoribonucleotide, this being obtained through hybridization of the sense RNA and anti-sense RNA strand for SEQ ID NO 2 and 14. SEQ ID NO 2 and 14 are a section of the cDNA of the interleukin-1 alpha. Both strands of the dsRNA exhibit continuously on the 3′ side two 2′-desoxythymidine residues each. The indications of quantity for anti-COX-2-dsRNA, anti-IL-6-dsRNA and anti-LOX-5-dsRNA refer to the total amount of dsRNA which is composed of the respective sequences (SEQ IDs) cited.

| | Dispersion No | | | | |
|---|---|---|---|---|---|
| | 1 | 2 | 3 | 4 | 5 |
| Polyoxyethylene(20)cetylstearyl ether | 1.00 | | | 0.5 | |
| Cetyl alcohol | | | 1.00 | | |
| Sodium polyacrylate | | 0.20 | | 0.30 | |
| Acrylate/C10-30 alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxy cinnamate | | | | 5.00 | 8.00 |
| 2,4-Bis-(4-(2-ethyl-hexyloxy-)2-hydroxyl)- | | 1.50 | | 2.00 | 2.50 |
| phenyl)-6-(4-methoxyphenyl)-(1,3,5)- | | | | | |
| triazine | | | | | |
| 1-(4-tert-Butyl phenyl)-3-(4- | 1.00 | | 2.00 | | |
| methoxyphenyl)-1,3-propanedione | | | | | |
| Diethylhexyl Butamido triazone | | 2.00 | | 2.00 | 1.00 |
| Ethylhexyl Triazone | 4.00 | | 3.00 | 4.00 | |
| 4-Methylbenzylidene camphor | 4.00 | 4.00 | | | 2.00 |
| Octocrylene | | 4.00 | 4.00 | | 2.50 |
| Phenylene-1,4-bis-(monosodium,2- | 1.00 | | 0.50 | | 2.00 |
| benzimidazyl-5,7-disulfonic acid | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 Alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea Butter | | 2.00 | | | |
| PVP hexadecane copolymer | 0.50 | | | 0.50 | 1.00 |
| Octoxyglycerin | | | 1.00 | | 0.50 |
| Glycerol | 3.00 | 7.50 | | 7.50 | 2.50 |
| Glycin soya | | | 1.50 | | |
| Tocopherol acetate | 0.50 | | 0.25 | | 1.00 |
| Anti-IL-1 alpha-dsRNA (dsRNA from SEQ | 0.10 | 0.10 | 0.10 | 0.10 | 0.10 |
| ID Nos 2 and 14) | | | | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad. 100 | ad. | ad. | ad. 100 | ad. |

### Example 5

### Production of a Gel Cream

Mixing the components cited in the table produced a gel cream of the composition also cited. The pH value of the gel cream was then set to 6.0.

Gel cream

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C12-15 Alkyl benzoate | 4.00 |
| Caprylic/Capric triglyceride | 3.00 |
| Cyclic dimethylpolysiloxane | 5.00 |
| Anti-IL-6-dsRNA (dsRNA from SEQ ID NOs | 0.10 |
| 48 and 51) | |
| Glycerol | 3.00 |
| Sodium hydroxide | q.s. |
| Preservative | q.s. |
| Perfume | q.s. |
| Water | ad 100.0 |

A gel cream was produced analogously using dsRNA, which was obtained through hybridization of the sequences SEQ NOs 53 and 57. The indications of quantity for anti-COX-2-dsRNA, anti-IL-6-dsRNA and anti-LOX-5-dsRNA refer to the total amount of dsRNA which is composed of the respective sequences (SEQ IDs) cited.

W/O Cream

### Example 6

### Production of a Cream Based on a Water-in-Oil Emulsion

Mixing the components cited in the table produced a cream of the composition also cited based on a water-in-01-dispersion.

| | |
|---|---|
| Polyglyceryl-3-diisostearate | 3.50 |
| Glycerol | 3.00 |
| Polyglyceryl-2-dipolyhydroxy stearate | 3.50 |
| Anti-IL-8-dsRNA (dsRNA from SEQ ID NOs | 0.10 |
| 60 and 66) | |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

An emulsion was produced analogously using dsRNA, which was obtained through hybridization of the sequences SEQ NOs 69 and 76. The indications of quantity for anti-COX-2-dsRNA, anti-IL-6-dsRNA und anti-LOX-5-dsRNA refer to the total amount of dsRNA which is composed of the respective sequences (SEQ IDs) cited.

W/OIW Cream

### Example 7

### Production of a Cream Based on a Water-in-Oil-in-Water Emulsion

Mixing the components cited in the table produced a cream of the composition also cited based on a water-in-oil-in-water dispersion. dsRNA was used as an oligoribonucleotide, this being obtained through hybridization of the sense RNA and anti-sense RNA strand for SEQ ID NO 30. SEQ ID NO 183 is a section of the cDNA of the cyclooxigenase. Both strands of the dsRNA exhibit continuously on the 3′ side two 2′-desoxythymidine residues each.

| | |
|---|---|
| Glyceryl stearate | 3.00 |
| PEG-100 stearate | 0.75 |
| Behenyl alcohol | 2.00 |
| Caprylic/Capric triglyceride | 8.0 |
| Octyldodecanol | 5.00 |
| C12-15 Alkyl benzoate | 3.00 |
| Anti-IL-Iβ-dsRNA (dsRNA from SEQ ID NO | 0.10 |
| 30) | |
| Magnesium sulfate (MgSO4) | 0.80 |
| Ethylenediaminetetraacetic acid | 0.10 |
| Preservative | q.s. |
| Perfume | q.s. |
| Water | ad 100.0 |

1. Double-stranded oligoribonucleotide, or a physiologically compatible salt thereof, which is capable of inducing the degradation of mRNA of one of more structures involved in inflammation or irritation of the skin.

2. Oligoribonucleotide according to claim 1, wherein the structures involved in inflammation or irritation of the skin are involved in the production of proinflammatory eicosanoides or cytokines.

3. Oligoribonucleotide according to claim 2, wherein the structures involved in inflammation or irritation of the skin are one or more of cyclooxygenase 2,5-lipoxygenase, or 5-lipoxygenase activating protein.

4. Oligoribonucleotide according to claim 2, wherein the structures involved in inflammation or irritation of the skin are one or more of interleukin-1 alpha or beta, interleukin-6, interleukin-8, or tumor necrosis factor alpha.

5. Oligoribonucleotide according to claim 1, wherein the oligoribonucleotide inhibits expression of the gene for the structure involved in inflammation or irritation of the skin by at least 30%.

6. Oligoribonucleotide according to claim 1, wherein the oligoribonucleotide inhibits expression of the gene for the structure involved in inflammation or irritation of the skin by at least 50%.

7. Oligoribonucleotide according to claim 1, wherein the oligoribonucleotide varies from the target sequence by 0 to 2 base pairs in relation to a length of 20 base pairs.

8. Oligoribonucleotide according to claim 1, wherein the oligoribonucleotide exhibits a length of 15 to 49 base pairs.

9. Oligoribonucleotide according to claim 1, wherein the oligoribonucleotide exhibits a length of 19 to 25 base pairs.

10. Oligoribonucleotide according to claim 1, wherein the oligoribonucleotide is homologous to a section of the gene of the structure involved in the inflammation or irritation of the skin, wherein the 5′ side is flanked by two adenosine residues and on the 3′ side by two thymidine residues.

11. Oligoribonucleotide accotding to claim 10, wherein the oligoribonucleotide is homologous to a section of the gene of the structure involved in the inflammation or irritation of the skin, wherein the 5′ side is flanked by two adenosine residues and on the 3′ by one thymidine and one cytosine residue.

12. Oligoribonucleotide according to claim 1, wherein the oligoribonucleotide bears two desoxythymidine residues on the 3′ end.

13. Oligoribonucleotide according to claim 1, wherein the oligoribonucleotide is singly integrated in an expression vector.

14. Oligoribonucleotide according to claim 1, wherein the oligoribonucleotide is multiply integrated in an expression vector.

15. Oligoribonucleotide according to claim 1, wherein one or more phosphate groups are replaced by a group selected from the group consisting of phosphothioate, methylphosphonate, and phosphoramidate.

16. Oligoribonucleotide according to claim 1, wherein one or more ribose residues are replaced by a residue selected from the group of amino acid residues and morpholine residues.

17. Oligoribonucleotide according to claim 1, wherein one or more ribose residues are modified by a residue selected from the group consisting of fluorine residues, alkyl residues, and O-alkyl residues.

18. Oligoribonucleotide according to claim 1, wherein the oligoribonucleotide contains one or more alpha nucleosides.

19. Pharmaceutical or cosmetic composition comprising one or more double-stranded oligoribonucleotide, or a physiologically compatible salt thereof, which is capable of inducing the degradation of mRNA of one of more structures involved in inflammation or irritation of the skin.

20. Composition according to claim 19, wherein the composition is formulated for topical administration.

21. Composition according to claim 18, wherein the composition comprises a plurality of oligoribonucleotides which inhibit the expression of a plurality of different structures involved in the inflammation or irritation of the skin.

22. Composition according to claim 18, wherein the composition comprises a plurality of oligoribonucleotides which have as their target a plurality of sequence regions of the same gene for the structures involved in the inflammation or irritation of the skin.

23. Composition according to claim 18, characterized wherein the composition comprises 0.00001 to 10 weight % of the oligoribonucleotide.

24. Composition according to claim 18, wherein the composition comprises 1 to 5 different oligoribonucleotides.

25. Composition according to claim 18, wherein the composition comprises only oligoribonucleotides which inhibit the expression of one or more of the structures involved in inflammation or irritation of the skin.

26. Composition according to claim 18, wherein the composition is formulated as a solution, cream, ointment, lotion, hydrodispersion, lipodispersion, emulsion, Pickering emulsion, gel, solid pencil, or an aerosol.

27. A method of treating an inflammation or irritation of the skin comprising applying one or more double-stranded oligoribonucleotide, or a physiologically compatible salt thereof, which is capable of inducing the degradation of mRNA of one of more structures involved in inflammation or irritation of the skin.

28. A method of preparing a cosmetic or therapeutic composition for topical administration comprising formulating the composition with one or more double-stranded oligoribonucleotide, or a physiologically compatible salt thereof, which is capable of inducing the degradation of mRNA of one of more structures involved in inflammation or irritation of the skin.


SEQUENCE LISTING (194 sequences)

SEQ ID NO: 1 - DNA, 23 bp, Homo sapiens
aaacctctaaaacatccaagctt

SEQ ID NO: 2 - DNA, 23 bp, Homo sapiens
aaggttctgaagaagagacggtt

SEQ ID NO: 3 - DNA, 23 bp, Homo sapiens
aatacaactttatgaggatcatc

SEQ ID NO: 4 - DNA, 23 bp, Homo sapiens
aattcatcctgaatgacgccctc

SEQ ID NO: 5 - DNA, 23 bp, Homo sapiens
aagtataattcgagccaatgatc

SEQ ID NO: 6 - DNA, 23 bp, Homo sapiens
aatctggatgaagcagtgaaatt

SEQ ID NO: 7 - DNA, 23 bp, Homo sapiens
aaattaccgtgattctaagaatc

SEQ ID NO: 8 - DNA, 23 bp, Homo sapiens
aactcacggcactaagaactatt

SEQ ID NO: 9 - DNA, 23 bp, Homo sapiens
aagcaagactactgggtgtgctt

SEQ ID NO: 10 - DNA, 23 bp, Homo sapiens
aaaccaggcgtaggtctggagtc

SEQ ID NO: 11 - DNA, 23 bp, Homo sapiens
aagaagctaaatcctttactgtt

SEQ ID NO: 12 - DNA, 23 bp, Homo sapiens
aaatcctttactgttagtcattt

SEQ ID NO: 13 - DNA, 23 bp, Homo sapiens
aaccaacactaacatataatgtt

SEQ ID NO: 14 - DNA, 23 bp, Homo sapiens
aatcattttaattattattcttc

SEQ ID NO: 15 - DNA, 23 bp, Homo sapiens
aattattattcttcataacaatt

SEQ ID NO: 16 - DNA, 23 bp, Homo sapiens
aagaaatatgcacaatagcagtt

SEQ ID NO: 17 - DNA, 23 bp, Homo sapiens
aagaagccacagacctaggattt

SEQ ID NO: 18 - DNA, 23 bp, Homo sapiens
aagttgctgatgaactcttaatc

SEQ ID NO: 19 - DNA, 23 bp, Homo sapiens
aagccttcctgccgcaacagttt

SEQ ID NO: 20 - DNA, 23 bp, Homo sapiens
aatacttctcgaagccgagcctc

SEQ ID NO: 21 - DNA, 23 bp, Homo sapiens
aaggcaaagcacgaaatgttatt

SEQ ID NO: 22 - DNA, 23 bp, Homo sapiens
aatgttattttttaattattatt

SEQ ID NO: 23 - DNA, 23 bp, Homo sapiens
aattattatttatatatgtattt

SEQ ID NO: 24 - DNA, 23 bp, Homo sapiens
aataaatgtacattaattacctt

SEQ ID NO: 25 - DNA, 23 bp, Homo sapiens
aagggcttcaggcaggccgcgtc

SEQ ID NO: 26 - DNA, 23 bp, Homo sapiens
aaaaagcttggtgatgtctggtc

SEQ ID NO: 27 - DNA, 23 bp, Homo sapiens
aagtggtgttctccatgtccttt

SEQ ID NO: 28 - DNA, 23 bp, Homo sapiens
aatgacaaaatacctgtggcctt

SEQ ID NO: 29 - DNA, 23 bp, Homo sapiens
aaatacctgtggccttgggcctc

SEQ ID NO: 30 - DNA, 23 bp, Homo sapiens
aaggaaaagaatctgtacctgtc

SEQ ID NO: 31 - DNA, 23 bp, Homo sapiens
aatctgtacctgtcctgcgtgtt

SEQ ID NO: 32 - DNA, 23 bp, Homo sapiens
aagaagaagatggaaaagcgatt

SEQ ID NO: 33 - DNA, 23 bp, Homo sapiens
aagatggaaaagcgatttgtctt

SEQ ID NO: 34 - DNA, 23 bp, Homo sapiens
aaatcaataacaagctggaattt

SEQ ID NO: 35 - DNA, 23 bp, Homo sapiens
aataacaagctggaatttgagtc

SEQ ID NO: 36 - DNA, 23 bp, Homo sapiens
aagcagaaaacatgcccgtcttc

SEQ ID NO: 37 - DNA, 23 bp, Homo sapiens
aagagagctgtacccagagagtc

SEQ ID NO: 38 - DNA, 23 bp, Homo sapiens
aagaaaccctctgtcattcgctc

SEQ ID NO: 39 - DNA, 23 bp, Homo sapiens
aaccgcttccctatttatttatt

SEQ ID NO: 40 - DNA, 23 bp, Homo sapiens
aatttggactggtgtgctctctt

SEQ ID NO: 41 - DNA, 23 bp, Homo sapiens
aaaatatataagctcagattatt

SEQ ID NO: 42 - DNA, 23 bp, Homo sapiens
aaatatataagctcagattattt

SEQ ID NO: 43 - DNA, 23 bp, Homo sapiens
aaatgagcaaatatcatactgtt

SEQ ID NO: 44 - DNA, 23 bp, Homo sapiens
aatgagcaaatatcatactgttc

SEQ ID NO: 45 - DNA, 23 bp, Homo sapiens
aaatatcatactgttcaatggtt

SEQ ID NO: 46 - DNA, 23 bp, Homo sapiens
aatatcatactgttcaatggttc

SEQ ID NO: 47 - DNA, 23 bp, Homo sapiens
aatggttctgaaataaacttctc

SEQ ID NO: 48 - DNA, 23 bp, Homo sapiens
aaagagaagctctatctcccctc

SEQ ID NO: 49 - DNA, 23 bp, Homo sapiens
aactccttctccacaagcgcctt

SEQ ID NO: 50 - DNA, 23 bp, Homo sapiens
aatgggcattccttcttctggtc

SEQ ID NO: 51 - DNA, 23 bp, Homo sapiens
aaatatgtgaagctgagttaatt

SEQ ID NO: 52 - DNA, 23 bp, Homo sapiens
aatatgtgaagctgagttaattt

SEQ ID NO: 53 - DNA, 23 bp, Homo sapiens
aagaagtaccacttgaaacattt

SEQ ID NO: 54 - DNA, 23 bp, Homo sapiens
aatggaaagtggctatgcagttt

SEQ ID NO: 55 - DNA, 23 bp, Homo sapiens
aaatggctaactttatacatatt

SEQ ID NO: 56 - DNA, 23 bp, Homo sapiens
aatggctaactttatacatattt

SEQ ID NO: 57 - DNA, 23 bp, Homo sapiens
aaagaaatatttatattgtattt

SEQ ID NO: 58 - DNA, 23 bp, Homo sapiens
aaaccaccggaaggaaccatctc

SEQ ID NO: 59 - DNA, 23 bp, Homo sapiens
aacctttccaccccaaatttatc

SEQ ID NO: 60 - DNA, 23 bp, Homo sapiens
aattcataaaaaaattcattctc

SEQ ID NO: 61 - DNA, 23 bp, Homo sapiens
aaaaattcattctctgtggtatc

SEQ ID NO: 62 - DNA, 23 bp, Homo sapiens
aagcaaatctacttcaacacttc

SEQ ID NO: 63 - DNA, 23 bp, Homo sapiens
aatacaagattcctggttaaatt

SEQ ID NO: 64 - DNA, 23 bp, Homo sapiens
aacatacttatatgtaaagtatt

SEQ ID NO: 65 - DNA, 23 bp, Homo sapiens
aataatttttaaatataaggatt

SEQ ID NO: 66 - DNA, 23 bp, Homo sapiens
aatttttaaatataaggattttc

SEQ ID NO: 67 - DNA, 23 bp, Homo sapiens
aagagaatatccgaactttaatt

SEQ ID NO: 68 - DNA, 23 bp, Homo sapiens
aatttcaggaattgaatgggttt

SEQ ID NO: 69 - DNA, 23 bp, Homo sapiens
aaaaatgatgggacaataaattt

SEQ ID NO: 70 - DNA, 23 bp, Homo sapiens
aaaatgatgggacaataaatttt

SEQ ID NO: 71 - DNA, 23 bp, Homo sapiens
aaattttgccataaagtcaaatt

SEQ ID NO: 72 - DNA, 23 bp, Homo sapiens
aattttgccataaagtcaaattt

SEQ ID NO: 73 - DNA, 23 bp, Homo sapiens
aaagtcaaatttagctggaaatc

SEQ ID NO: 74 - DNA, 23 bp, Homo sapiens
aatttagctggaaatcctggatt

SEQ ID NO: 75 - DNA, 23 bp, Homo sapiens
aagtccttgttccactgtgcctt

SEQ ID NO: 76 - DNA, 23 bp, Homo sapiens
aagtgtaacttattaacctattt

SEQ ID NO: 77 - DNA, 23 bp, Homo sapiens
aacttattaacctatttattatt

SEQ ID NO: 78 - DNA, 23 bp, Homo sapiens
aacctatttattatttatgtatt

SEQ ID NO: 79 - DNA, 23 bp, Homo sapiens
aaatagaagatgaatcattgatt

SEQ ID NO: 80 - DNA, 23 bp, Homo sapiens
aaagatgttatagtaaatttatt

SEQ ID NO: 81 - DNA, 23 bp, Homo sapiens
aagatgttatagtaaatttattt

SEQ ID NO: 82 - DNA, 23 bp, Homo sapiens
aaatttattttattttagatatt

SEQ ID NO: 83 - DNA, 23 bp, Homo sapiens
aatgatgttttattagataaatt

SEQ ID NO: 84 - DNA, 23 bp, Homo sapiens
aagaaacaattgggtacccagtt

SEQ ID NO: 85 - DNA, 23 bp, Homo sapiens
aattgggtacccagttaaatttt

SEQ ID NO: 86 - DNA, 23 bp, Homo sapiens
aagttttaattgaactaacaatc

SEQ ID NO: 87 - DNA, 23 bp, Homo sapiens
aattgaactaacaatcctagttt

SEQ ID NO: 88 - DNA, 23 bp, Homo sapiens
aactattaaaacagccaaaactc

SEQ ID NO: 89 - DNA, 23 bp, Homo sapiens
aaaacagccaaaactccacagtc

SEQ ID NO: 90 - DNA, 23 bp, Homo sapiens
aaatagattcttataatattatt

SEQ ID NO: 91 - DNA, 23 bp, Homo sapiens
aatagattcttataatattattt

SEQ ID NO: 92 - DNA, 23 bp, Homo sapiens
aatattatttaaatgactgcatt

SEQ ID NO: 93 - DNA, 23 bp, Homo sapiens
aattttttttactgtttctgatt

SEQ ID NO: 94 - DNA, 23 bp, Homo sapiens
aaacatttaaaatataatttgtt

SEQ ID NO: 95 - DNA, 23 bp, Homo sapiens
aagcacctgctggacaagccctt

SEQ ID NO: 96 - DNA, 23 bp, Homo sapiens
aactgggcgagatccagctggtc

SEQ ID NO: 97 - DNA, 23 bp, Homo sapiens
aagcgatggagaacctgttcatc

SEQ ID NO: 98 - DNA, 23 bp, Homo sapiens
aacacatctggtgtctgaggttt

SEQ ID NO: 99 - DNA, 23 bp, Homo sapiens
aaagaagacatcccctactactt

SEQ ID NO: 100 - DNA, 23 bp, Homo sapiens
aagaagacatcccctactacttc

SEQ ID NO: 101 - DNA, 23 bp, Homo sapiens
aagtcctcaggcttccccaagtc

SEQ ID NO: 102 - DNA, 23 bp, Homo sapiens
aagcagctgccatattactactt

SEQ ID NO: 103 - DNA, 23 bp, Homo sapiens
aatcacgaccactgatagatgtc

SEQ ID NO: 104 - DNA, 23 bp, Homo sapiens
aagaaactgtaggcaatgttgtc

SEQ ID NO: 105 - DNA, 23 bp, Homo sapiens
aaacgcatcatactcttcctgtt

SEQ ID NO: 106 - DNA, 23 bp, Homo sapiens
aacgcatcatactcttcctgttc

SEQ ID NO: 107 - DNA, 23 bp, Homo sapiens
aactattacctcatcttcttttt

SEQ ID NO: 108 - DNA, 23 bp, Homo sapiens
aagacgatctccaccaccatctc

SEQ ID NO: 109 - DNA, 23 bp, Homo sapiens
aactctctgctgaatatggggtt

SEQ ID NO: 110 - DNA, 23 bp, Homo sapiens
aatatggggttggtgttctcatc

SEQ ID NO: 111 - DNA, 23 bp, Homo sapiens
aatacctacaagtcatcataatt

SEQ ID NO: 112 - DNA, 23 bp, Homo sapiens
aaatctattggccatctgggctt

SEQ ID NO: 113 - DNA, 23 bp, Homo sapiens
aatctattggccatctgggcttc

SEQ ID NO: 114 - DNA, 23 bp, Homo sapiens
aacatgaccgtggccccaaattt

SEQ ID NO: 115 - DNA, 23 bp, Homo sapiens
aaatttgctattcccatgcattt

SEQ ID NO: 116 - DNA, 23 bp, Homo sapiens
aatttgctattcccatgcatttt

SEQ ID NO: 117 - DNA, 23 bp, Homo sapiens
aaaataaaatgcagagacatgtt

SEQ ID NO: 118 - DNA, 23 bp, Homo sapiens
aaataaaatgcagagacatgttt

SEQ ID NO: 119 - DNA, 23 bp, Homo sapiens
aataaaatgcagagacatgtttt

SEQ ID NO: 120 - DNA, 23 bp, Homo sapiens
aactcctcagcagcgcctccttc

SEQ ID NO: 121 - DNA, 23 bp, Homo sapiens
aaaactgctcaacaccggaattt

SEQ ID NO: 122 - DNA, 23 bp, Homo sapiens
aactgctcaacaccggaattttt

SEQ ID NO: 123 - DNA, 23 bp, Homo sapiens
aaaattatttctgaaacccactc

SEQ ID NO: 124 - DNA, 23 bp, Homo sapiens
aacgttgtgaataacattccctt

SEQ ID NO: 125 - DNA, 23 bp, Homo sapiens
aatgcaattatgagttatgtgtt

SEQ ID NO: 126 - DNA, 23 bp, Homo sapiens
aaattgcttctaagaagaaagtt

SEQ ID NO: 127 - DNA, 23 bp, Homo sapiens
aattgcttctaagaagaaagttc

SEQ ID NO: 128 - DNA, 23 bp, Homo sapiens
aagaagaaagttcatccctgatc

SEQ ID NO: 129 - DNA, 23 bp, Homo sapiens
aaatcatatttacggtgaaactc

SEQ ID NO: 130 - DNA, 23 bp, Homo sapiens
aattgatggagagatgtatcctc

SEQ ID NO: 131 - DNA, 23 bp, Homo sapiens
aagatactcaggcagagatgatc

SEQ ID NO: 132 - DNA, 23 bp, Homo sapiens
aagtccctgagcatctacggttt

SEQ ID NO: 133 - DNA, 23 bp, Homo sapiens
aatggggtgatgagcagttgttc

SEQ ID NO: 134 - DNA, 23 bp, Homo sapiens
aatggggtgatgagcagttgttc

SEQ ID NO: 135 - DNA, 23 bp, Homo sapiens
aatggggtgatgagcagttgttc

SEQ ID NO: 136 - DNA, 23 bp, Homo sapiens
aacacttgagtggctatcacttc

SEQ ID NO: 137 - DNA, 23 bp, Homo sapiens
aaatttgacccagaactactttt

SEQ ID NO: 138 - DNA, 23 bp, Homo sapiens
aatttgacccagaactacttttc

SEQ ID NO: 139 - DNA, 23 bp, Homo sapiens
aactacttttcaacaaacaattc

SEQ ID NO: 140 - DNA, 23 bp, Homo sapiens
aattccagtaccaaaatcgtatt

SEQ ID NO: 141 - DNA, 23 bp, Homo sapiens
aaaatcgtattgctgctgaattt

SEQ ID NO: 142 - DNA, 23 bp, Homo sapiens
aatacaactatcaacagtttatc

SEQ ID NO: 143 - DNA, 23 bp, Homo sapiens
aacatggaattacccagtttgtt

SEQ ID NO: 144 - DNA, 23 bp, Homo sapiens
aaaagcctcggccagatgccatc

SEQ ID NO: 145 - DNA, 23 bp, Homo sapiens
aagcctcggccagatgccatctt

SEQ ID NO: 146 - DNA, 23 bp, Homo sapiens
aaatcatcaacactgcctcaatt

SEQ ID NO: 147 - DNA, 23 bp, Homo sapiens
aatcatcaacactgcctcaattc

SEQ ID NO: 148 - DNA, 23 bp, Homo sapiens
aataacgtgaagggctgtccctt

SEQ ID NO: 149 - DNA, 23 bp, Homo sapiens
aagggctgtccctttacttcatt

SEQ ID NO: 150 - DNA, 23 bp, Homo sapiens
aacagtcaccatcaatgcaagtt

SEQ ID NO: 151 - DNA, 23 bp, Homo sapiens
aagtctaatgatcatatttattt

SEQ ID NO: 152 - DNA, 23 bp, Homo sapiens
aatttaattatttaataatattt

SEQ ID NO: 153 - DNA, 23 bp, Homo sapiens
aattatttaataatatttatatt

SEQ ID NO: 154 - DNA, 23 bp, Homo sapiens
aactccttatgttacttaacatc

SEQ ID NO: 155 - DNA, 23 bp, Homo sapiens
aagtttggaaaacagtttttatt

SEQ ID NO: 156 - DNA, 23 bp, Homo sapiens
aaagtaaagatgtttgaatactt

SEQ ID NO: 157 - DNA, 23 bp, Homo sapiens
aagatgccaaaatgctgaaagtt

SEQ ID NO: 158 - DNA, 23 bp, Homo sapiens
aaagtttttacactgtcgatgtt

SEQ ID NO: 159 - DNA, 23 bp, Homo sapiens
aagtttttacactgtcgatgttt

SEQ ID NO: 160 - DNA, 23 bp, Homo sapiens
aagtaactaatgtttgaaatttt

SEQ ID NO: 161 - DNA, 23 bp, Homo sapiens
aacaaaacaggtatcagtgcatt

SEQ ID NO: 162 - DNA, 23 bp, Homo sapiens
aaaacaggtatcagtgcattatt

SEQ ID NO: 163 - DNA, 23 bp, Homo sapiens
aatgaatatttaaattagacatt

SEQ ID NO: 164 - DNA, 23 bp, Homo sapiens
aatgaatatttaaattagacatt

SEQ ID NO: 165 - DNA, 23 bp, Homo sapiens
aattagacattaccagtaatttc

SEQ ID NO: 166 - DNA, 23 bp, Homo sapiens
aaatcagcaatgaaacaataatt

SEQ ID NO: 167 - DNA, 23 bp, Homo sapiens
aatcagcaatgaaacaataattt

SEQ ID NO: 168 - DNA, 23 bp, Homo sapiens
aatgaaacaataatttgaaattt

SEQ ID NO: 169 - DNA, 23 bp, Homo sapiens
aataatttgaaatttctaaattc

SEQ ID NO: 170 - DNA, 23 bp, Homo sapiens
aatctgtaaaatcagatgaaatt

SEQ ID NO: 171 - DNA, 23 bp, Homo sapiens
aaattttactacaattgcttgtt

SEQ ID NO: 172 - DNA, 23 bp, Homo sapiens
aaaatattttataagtgatgttc

SEQ ID NO: 173 - DNA, 23 bp, Homo sapiens
aagaatatcctgttgagatattc

SEQ ID NO: 174 - DNA, 23 bp, Homo sapiens
aaaggagaaaagcccaaattatt

SEQ ID NO: 175 - DNA, 23 bp, Homo sapiens
aaactttttgaagcaaacttttt

SEQ ID NO: 176 - DNA, 23 bp, Homo sapiens
aactttttgaagcaaactttttt

SEQ ID NO: 177 - DNA, 23 bp, Homo sapiens
aacgatatgttttctcagatttt

SEQ ID NO: 178 - DNA, 23 bp, Homo sapiens
aatttagcagtccatatcacatt

SEQ ID NO: 179 - DNA, 23 bp, Homo sapiens
aatgacctcataaaatacctctt

SEQ ID NO: 180 - DNA, 23 bp, Homo sapiens
aaattcatttcacacattaattt

SEQ ID NO: 181 - DNA, 23 bp, Homo sapiens
aattcatttcacacattaatttt

SEQ ID NO: 182 - DNA, 23 bp, Homo sapiens
aattcagtaggtgcattggaatc

SEQ ID NO: 183 - DNA, 23 bp, Homo sapiens
aaacacttcgtttctcctatttt

SEQ ID NO: 184 - DNA, 23 bp, Homo sapiens
aaattagacattaccagtaattt

SEQ ID NO: 185 - DNA, 23 bp, Homo sapiens
aaacaaccctcagacgccacatc

SEQ ID NO: 186 - DNA, 23 bp, Homo sapiens
aagctgccaggcaggttctcttc

SEQ ID NO: 187 - DNA, 23 bp, Homo sapiens
aagacaggggggccccagggctc

SEQ ID NO: 188 - DNA, 23 bp, Homo sapiens
aatcggcccgactatctcgactt

SEQ ID NO: 189 - DNA, 23 bp, Homo sapiens
aatccctttattaccccctcctt

SEQ ID NO: 190 - DNA, 23 bp, Homo sapiens
aagagaattgggggcttagggtc

SEQ ID NO: 191 - DNA, 23 bp, Homo sapiens
aactcactggggcctacagcttt

SEQ ID NO: 192 - DNA, 23 bp, Homo sapiens
aatctggagaccagggagccttt

SEQ ID NO: 193 - DNA, 23 bp, Homo sapiens
aatatttatctgattaagttgtc

SEQ ID NO: 194 - DNA, 23 bp, Homo sapiens
aagttgtctaaacaatgctgatt